(19) United States
(12) Patent Application Publication
(10) Pub. No.: US 2026/0315092 A1
(43) Pub. Date: Oct. 15, 2026

(54) ENDOSCOPIC SURGICAL INSTRUMENT HAVING A RETRACTABLE CUTTING BLADE

(71) Applicant: A.M. SURGICAL, INC., Nashua, NH (US)

(72) Inventor: Romi Mirza, Las Vegas, NV (US)

(21) Appl. No.: 19/633,034

(22) Filed: Mar. 30, 2026

Related U.S. Application Data
(60) Provisional application No. 63/780,678, filed on Mar. 31, 2025.

(51) Int. Cl. A61B 17/32; A61B 1/05; A61B 1/317; A61B 17/00

(52) U.S. Cl. CPC A61B 17/320036; A61B 1/05; A61B 1/317; A61B 2017/00477

(57) ABSTRACT

An endoscopic surgical instrument has a main housing and a spring-biased lever pivotally mounted on the main housing, as well as a cannula having a slot and an inner tube disposed within a lumen of the cannula. The inner tube has a bore, and a side wall in which a slot is formed. Within the inner tube bore is situated a blade assembly having a retractable cutting blade. An inner slide member is reciprocatingly moveable within the inner tube bore and coupled to the lever and the blade assembly such that pivotal movement of the lever on the main housing causes the cutting blade either to extend outwardly from the surgical instrument through the inner tube bore and cannula slot to effect tissue cutting during a surgical procedure or to retract safely into the inner tube bore without projecting therefrom and from the slot of the cannula.

### CROSS-REFERENCE TO RELATED APPLICATIONS

This application is related to U.S. Provisional Patent Application Ser. No. 63/780,678, filed on Mar. 31, 2025, and titled “Endoscopic Surgical Instrument Having a Retractable Cutting Blade”, the disclosure of which is hereby incorporated by reference and on which priority is hereby claimed.

### BACKGROUND OF THE INVENTION

### Field of the Invention

The present invention generally relates to medical devices, and more particularly relates to devices and methods for endoscopic surgery. Even more specifically, this invention relates to procedures and endoscopic surgical instruments used in carpal tunnel or pulley release surgery, or any soft tissue release surgery (e.g., cubital tunnel and gastroc releases).

### Description of the Prior Art

Endoscopic surgery is a minimally invasive surgical procedure that is performed through small incisions or natural body openings. An endoscopic procedure typically involves use of specialized devices and remote-control manipulation of instruments with indirect observation of the surgical field through an endoscope or similar device. Comparing to open surgery, endoscopic surgery may result in shorter hospital stays, or allow outpatient treatment.

Carpal tunnel syndrome is an entrapment median neuropathy resulting from compression of the median nerve at the wrist in the carpal tunnel. Symptoms of carpal tunnel syndrome include tingling, numbness, weakness, or pain felt in the fingers supplied by the median nerve or in the palm. Repetitive tasks, force, posture, and vibration have been cited as causative or contributing factors to carpal tunnel syndrome. Palliative treatments for carpal tunnel syndrome include direct corticosteroid injections, splinting, oral corticosteroids and/or behavior modification.

Typically, endoscopic surgery has involved a number of steps and separate devices for performing tendon, pulley or tunnel division. After making an incision and opening a path to the pulley or tunnel using a blunt instrument, a cannula is inserted into the path. Briefly, in order to smoothly insert the cannula, the central lumen of the cannula must be filled with a device, such as an obturator. The obturator is then removed and an endoscope, or arthroscope, is inserted into the cannula to view the pulley or tunnel. The endoscope is then withdrawn from the cannula, a knife is either advanced into the cannula for division or a specialized knife assembly is affixed to the endoscope and the knife/endoscope assembly is advanced into the cannula for division.

A.M. Surgical, Inc., the applicant herein, has developed and patented numerous compact surgical instruments for uniportal endoscopic pulley or tunnel release surgery that eliminate the need for a separate device, such as an obturator, for filling the cannula during insertion and eliminate the need to remove the endoscope in order to insert a cutting blade or blade assembly. Such instruments have revolutionized and simplified carpal tunnel surgical procedures, in particular, and many other surgical procedures, in general (for example, uniportal plantar fascia release, lateral release for patella realignment, release of the posterior and other compartments of the leg, and forearm fascia release for fascial compartment syndrome, and other endoscopic surgical divisions or partial separation of a tendon or ligament, cutting, dividing, separating or making an incision in connective tissue, muscle, cartilage, membranes, skin, other body tissues or organs, and other procedures).

One such endoscopic surgical instrument is disclosed in U.S. Pat. No. 8,911,470 (Mirza et al.), assigned of record to A.M. Surgical, Inc., the disclosure of which is incorporated herein by reference. The instrument includes a slotted clear (transparent) cannula, a cutting blade and a housing to which the slotted cannula is attached. The blade is “parked” within the instrument housing in a revolver assembly until deployed. The revolver assembly may be rotated to position the cutting blade in alignment with the cannula and, by doing so, the blade mounts to the distal end of a guidance tube passing through the housing and the revolver assembly. The guidance tube, with the cutting blade affixed to the end thereof, is advanced on the housing through the lumen of the cannula. The cutting blade protrudes through the cannula slot as it advances thereon from the proximal end to the distal end thereof such that any tissue or ligaments exposed to the cutting blade during a surgical procedure will be cut or severed. Other patents disclosing compact endoscopic surgical instruments that are assigned of record to A.M. Surgical include U.S. Pat. Nos. 8,979,880; 9,066,746; 9,179,930; 9,211,136; 9,408,623; 9,445,830; 9,610,089; 9,808,274; 9,931,133; 10,265,093; and 10,548,624, the disclosure of each of which is incorporated herein by reference.

The surgical instruments disclosed in the aforementioned patents work fine for their intended purpose, but each of these instruments has its cutting blade always protruding through the cannula slot when moved into the cannula. Thus, during a surgical procedure, when the cannula is properly positioned at the surgical site, any tissue or ligament in proximity to the cannula will be exposed to the cutting blade and will be cut or severed as the blade is advanced or retracted on the cannula.

The applicant herein is also the record owner by assignment of U.S. Pat. No. 11,596,430 (Mirza, et al.) and U.S. Pat. No. 12,268,407 (Mirza), each patent being titled “Endoscopic Surgical Instrument Having a Retractable Cutting Blade and Surgical Procedure Using Same”, the disclosure of each which is incorporated herein by reference. In each of the aforementioned patents, a surgical instrument is disclosed which has a blade assembly which includes a retractable cutting blade. Each of the surgical instruments is capable of receiving an endoscope or arthroscope for viewing tissue at a surgical site during a surgical procedure. The distal end portion of the endoscope or arthroscope operatively engages the blade assembly such that relative movement between the endoscope or arthroscope and the surgical instrument triggers the cutting blade to either be exposed on the surgical instrument in a tissue cutting position or be retracted safely into the surgical instrument to prevent the inadvertent cutting of tissue during a surgical procedure.

### OBJECTS AND SUMMARY OF THE INVENTION

It is an object of the present invention to provide an endoscopic surgical instrument having a retractable cutting blade.

It is another object of the present invention to provide an endoscopic surgical instrument having a retractable cutting blade sharpened at both edges which can perform an antegrade (forward) and retrograde (reverse) cut at the choice of the user.

It is a further object of the present invention to provide an endoscopic surgical instrument having a cannula formed with an axially-extending slot from which a cutting blade may be selectively caused to protrude or be retracted into the cannula at a selected position along at least a portion of the cannula.

It is a further object of the present invention to provide an endoscopic surgical instrument having a cutting blade situated in an inner tube that is received in the lumen of a slotted cannula, the blade being biased by a spring coupled to a lever situated on the handle or main housing of the instrument to be in a retracted state within the tube and selectively positionable in a projected state in which the blade protrudes from the tube and the slotted cannula when the lever arm is moved against the bias of the spring.

It is yet a further object of the present invention to provide an endoscopic surgical instrument having a retractable cutting blade and a cannula formed with an axially-extending slot from which the blade may be selectively caused to project, and which the instrument cooperates with an endoscope or arthroscope.

It is another object of the present invention to provide a surgical instrument which is attachable to an endoscope or arthroscope and having a retractable cutting blade, and which may be easily manipulated by a surgeon during a surgical procedure and used either for viewing the surgical site with the endoscope and with the blade retracted or for viewing with the endoscope while simultaneously cutting tissue with the retractable blade at the surgical site.

In accordance with one form of the present invention, an endoscopic surgical instrument includes a main housing having mateable front and back housing sections, a spring-biased lever mounted on the main housing, a generally tubular front slide section which passes through the main housing and in which is receivable the tubular distal section of an endoscope or arthroscope for viewing a surgical site by a surgeon performing a surgical procedure on a patient, a transparent, slotted cannula, and a back cannula section to which the transparent cannula is affixed, the back cannula section being removably mounted to the main housing, and a clamp ring which is partially rotatable on the front slide section to secure an endoscope or arthroscope in place and in a desired position on and relative to the surgical instrument. The surgical instrument further includes an inner tube which extends through the lumen of the transparent cannula and includes a slot formed through the wall thereof near the distal end of the inner tube, the slot of the inner tube being in alignment with the slot of the transparent cannula.

An elongated, inner slide member resides within the axial bore of the inner tube and is axially movable therein. The inner slide member is joined to the lever and reciprocatingly moves within the bore of the inner tube with pivotal movement of the lever. Advantageously, the inner slide member is particularly shaped so as not to interfere with the placement and positioning of the tubular distal end portion of the endoscope or arthroscope also situated within the bore of the inner tube. The inner tube has a cutout formed in the wall thereof near the distal end thereof, which cutout defines a window through which, in addition to the transparent cannula, the camera or viewing sensor of the endoscope or arthroscope may view the surgical site during a surgical procedure.

The bore of the inner tube also houses a retractable blade assembly near the distal end of the tube. The retractable blade assembly is operatively coupled to the inner slide member, and has a cutting blade. When the lever on the main housing is pivoted against the bias of the spring to a first position, the inner slide member also moves in a first axial direction within the inner tube, causing the cutting blade to project not only through the slot formed in the wall of the inner tube but also through the slot of the cannula to effect a tissue cutting procedure. When the lever is moved to a second position opposite the first position, the inner slide member concomitantly moves in a second axial direction opposite the first direction to cause the cutting blade to safely retract into the bore of the inner tube without projecting therefrom or the slotted cannula.

These and other objects, features and advantages of the present invention will be apparent from the following detailed description of illustrative embodiments thereof, which is to be read in connection with the accompanying drawings.

### BRIEF DESCRIPTION OF THE DRAWINGS

FIG. 1 is a perspective view of an endoscopic surgical instrument formed in accordance with the present invention, and illustrating the cutting blade thereof in a retracted state.

FIG. 2 is a perspective view of the endoscopic surgical instrument formed in accordance with the present invention, and illustrating the cutting blade thereof in a projected state.

FIG. 3 is a longitudinal cross-sectional view of the endoscopic surgical instrument of the present invention, and illustrating the cutting blade thereof in a projected state, the back cannula of the instrument being shown partially separated from the main housing of the instrument.

FIG. 3A is an enlarged, longitudinal cross-sectional view of the distal end portion of the endoscopic surgical instrument of the present invention shown in FIG. 3.

FIG. 4 is an exploded view of the endoscopic surgical instrument of the present invention.

FIG. 5 is a side elevational view of the endoscopic surgical instrument of the present invention, the back cannula of the instrument being shown partially separated from the main housing of the instrument.

FIGS. 6A-6G are various views of the inner tube forming a component of the endoscopic surgical instrument of the present invention.

FIGS. 7A-7F are various views of the pivot cap forming a component of the retractable blade assembly of the endoscopic surgical instrument of the present invention.

FIGS. 8A-8F are various views of the inner slide member forming a component of the endoscopic surgical instrument of the present invention.

FIGS. 9A-9E are various views of the front slide forming a component of the endoscopic surgical instrument of the present invention.

FIGS. 10A-10D are additional various views of the front slide forming a component of the endoscopic surgical instrument of the present invention.

FIGS. 11A-11D are various views of the clamp ring forming a component of the endoscopic surgical instrument of the present invention.

FIGS. 12A-12J are additional various views of the clamp ring forming a component of the endoscopic surgical instrument of the present invention.

FIGS. 13A-13C are various views of the lever forming a component of the endoscopic surgical instrument of the present invention.

FIGS. 14A-14F are various views of the front housing section forming a component of the main housing of the endoscopic surgical instrument of the present invention.

FIGS. 15A-15H are additional various views of the front housing forming a component of the main housing of the endoscopic surgical instrument of the present invention.

FIGS. 16A-16E are various views of the inner slide assembly forming a component of the endoscopic surgical instrument of the present invention.

FIGS. 17A-17D are various views of the yoke forming a component of the retractable blade assembly of the endoscopic surgical instrument of the present invention.

FIGS. 18A-18D are various views of the cannula forming a component of the endoscopic surgical instrument of the present invention.

FIGS. 19A-19F are various views of the back housing section forming a component of the main housing of the endoscopic surgical instrument of the present invention.

FIGS. 20A-20H are additional various views of the back housing section forming a component of the main housing of the endoscopic surgical instrument of the present invention.

FIGS. 21A-21E are various views of the back cannula forming a component of the endoscopic surgical instrument of the present invention.

FIGS. 22A-22I are additional various views of the back cannula forming a component of the endoscopic surgical instrument of the present invention.

### DETAILED DESCRIPTION OF THE PREFERRED EMBODIMENTS

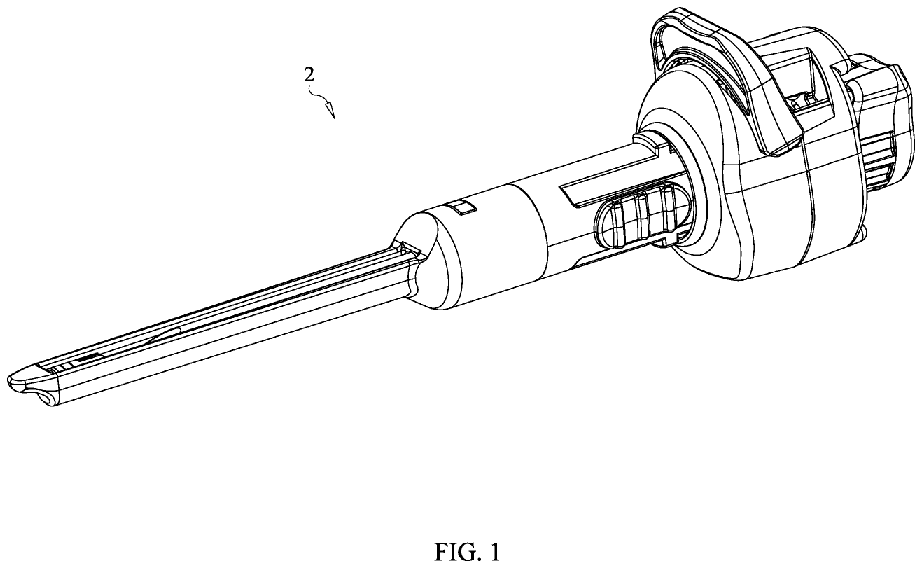

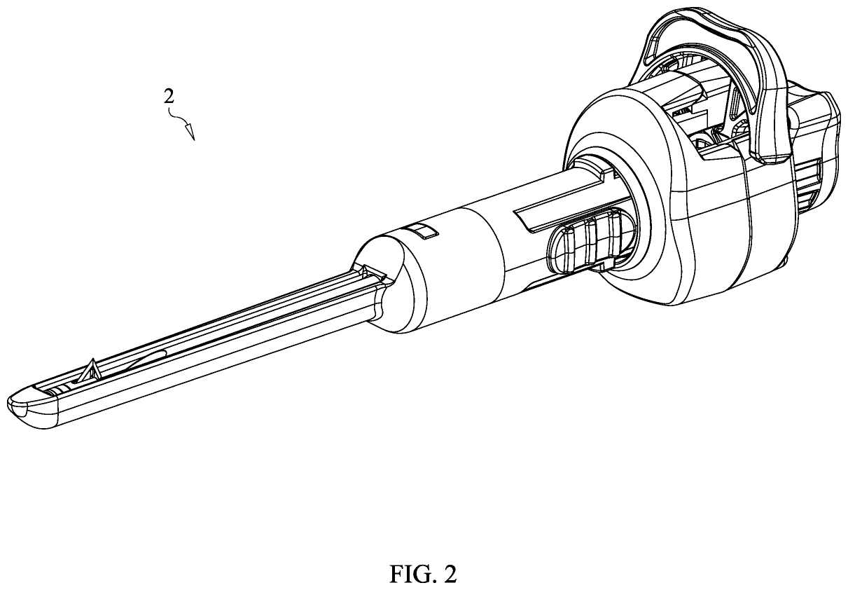

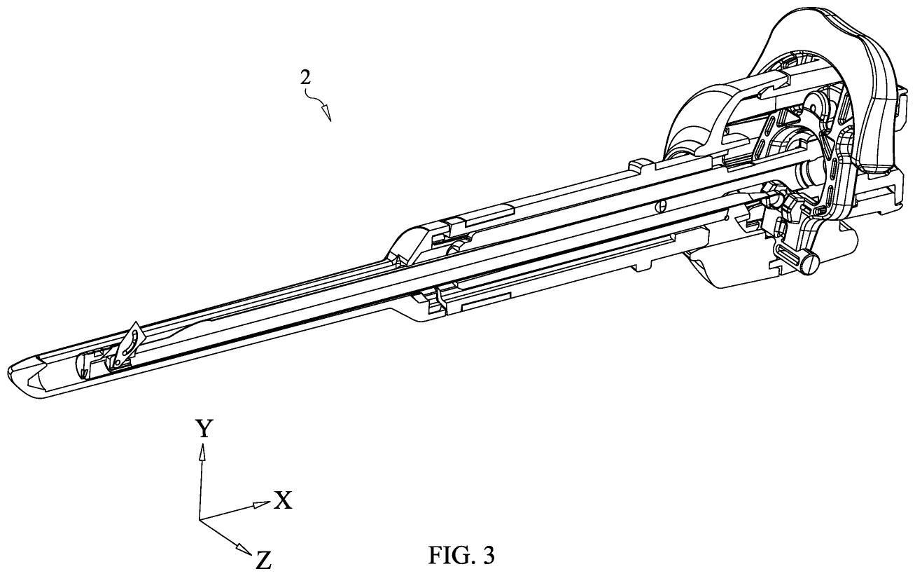

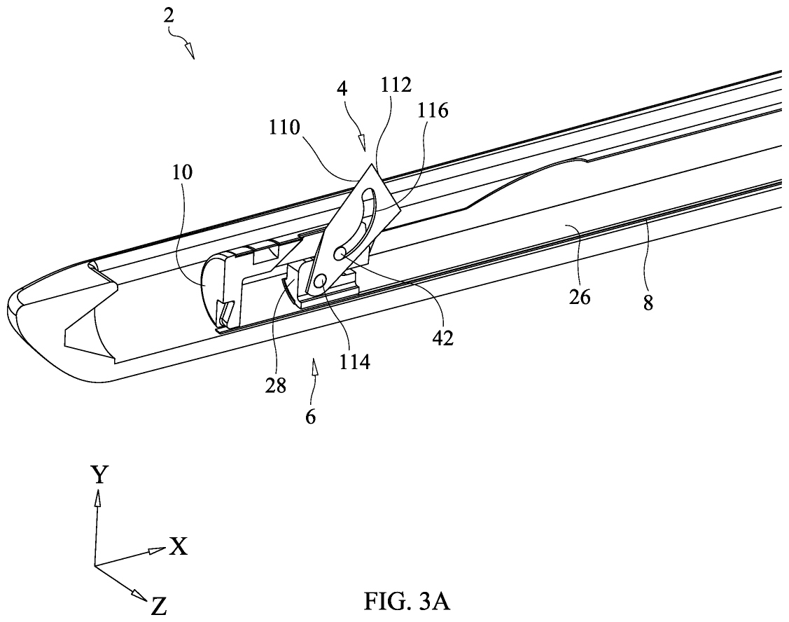

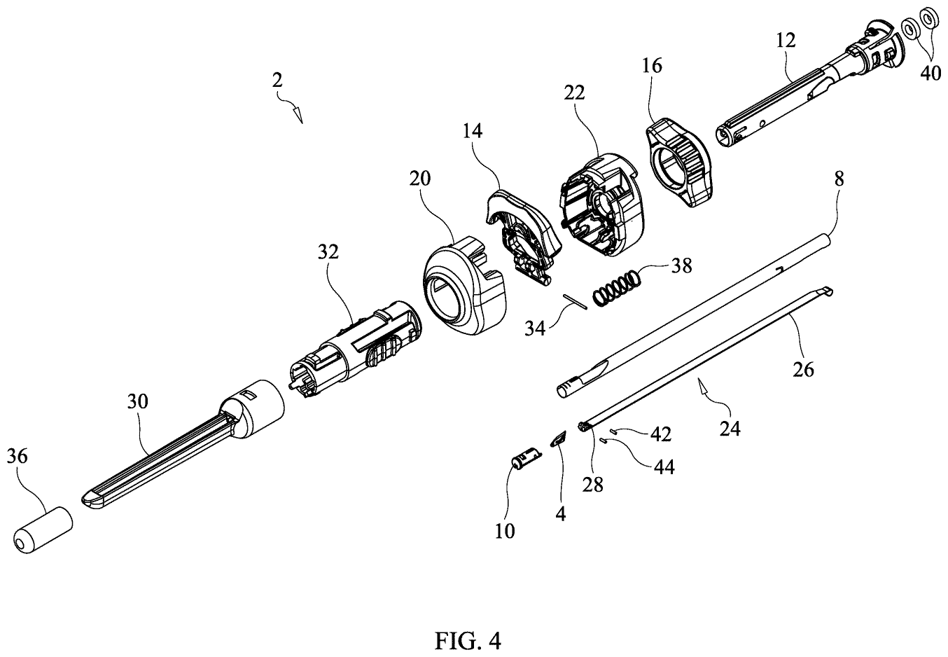

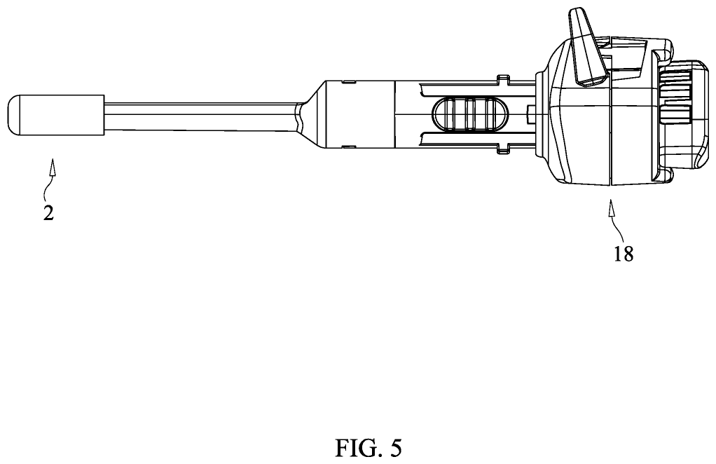

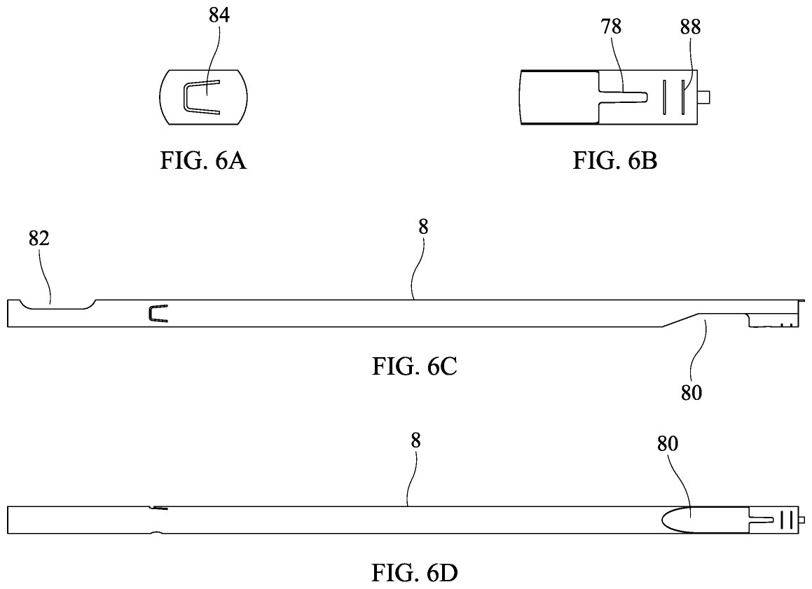

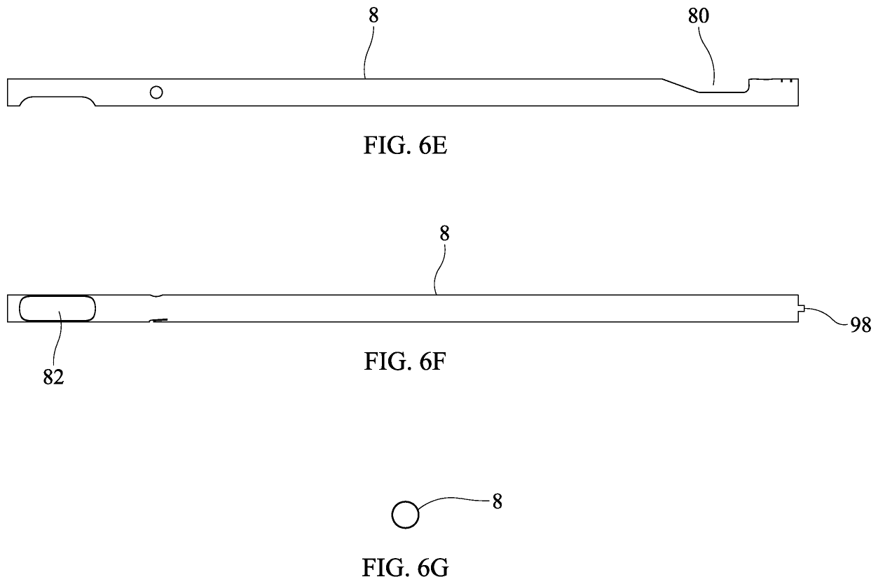

Reference should initially be had to FIGS. 1-5 of the drawings, which illustrate one form of an endoscopic surgical instrument 2 constructed in accordance with the present invention. More specifically, FIG. 1 shows the endoscopic surgical instrument 2 with the cutting blade 4 retracted, whereas FIG. 2 shows the endoscopic surgical instrument 2 with the cutting blade 4 projecting outwardly therefrom. FIG. 3 shows various components of the endoscopic surgical instrument 2 of the present invention by way of a longitudinal cross-sectional view. FIG. 4 is an exploded view of the endoscopic surgical instrument 2 and the components thereof, and FIG. 5 is a side view of the endoscopic surgical instrument 2. As can be seen from these figures, the endoscopic surgical instrument 2 of the present invention includes a retractable cutting blade 4 forming part of a retractable blade assembly 6, an inner tube 8, a pivot cap 10, a front slide 12, a lever 14, a clamp ring 16, a front housing section 20, an inner slide assembly 24 comprised of an inner slide member 26 and a yoke 28, the yoke 28 also forming part of the retractable blade assembly 6, a cannula 30, a back cannula section 32, a back housing section 22, a cross pin 34, a protective end cap 36, a compression spring 38, a pair of O-rings 40 and an actuator pin 42 and a pivot pin 44 forming parts of the retractable blade assembly 6. Most if not all of these components are also shown individually in the various views of FIGS. 6A-22I.

As used herein, the term “distal end portion” refers to structures of components of the surgical instrument 2 of the present invention which are relatively closer to the obturator end 62 of the cannula 30 of the surgical instrument 2 than structures of components referred to by the term “proximal end portion” and may encompass significant portions of the components.

Similarly, as used herein, the term “proximal end portion” refers to structures of components of the surgical instrument 2 of the present invention which are relatively closer to the end of the surgical instrument 2 where the clamp ring 16 is located than structures of components referred to by the term “distal end portion” and may encompass significant portions of the components.

Still with reference to FIGS. 1-5 of the drawings, it will be seen that the endoscopic surgical instrument 2 of the present invention includes a main housing 18 which is defined by the front housing section 20 and the back housing section 22 when the two sections are mated together. A lever 14 straddles the main housing 18 and is biased by a compression spring 38 in a forward direction on the housing to maintain the cutting blade 4 in a retracted state on the surgical instrument 2, as will be explained in greater detail. To cause the cutting blade 4 to extend outwardly to a projected state to effect a tissue cutting procedure, the surgeon exerts finger pressure on the lever 14 to move the lever 14 in a rearward position on the main housing 18 of the instrument against the bias of the spring 38.

The front slide 12 includes a tubular section 46 on the distal end portion thereof. This tubular section 46, as well as a portion of the proximal end portion of the front slide 12, is received by the main housing 18 and passes through aligned openings formed in walls of the back housing section 22 and the front housing section 20 such that the tubular distal end portion of the front slide 12 extends outwardly from the front housing section 20. The tubular distal end portion of the front slide 12 includes a bore which fixedly receives the proximal end portion of the inner tube 8.

The proximal end portion of the front slide 12 is partially split into preferably three conjoined sections 48 such that the split rear section 50 thereof is resilient and compressible to thereby change the outer diameter as well as the inner diameter of the bore defined by the split rear section 50 of the front slide 12. Preferably, one or two resilient O-rings 40 are mounted within the bore defined by the split rear section 50 of the proximal end portion of the front slide 12. A clamp ring 16 is mounted on the proximal end portion of the front slide 12 over the split rear section 50 thereof and is partially rotatable thereon. The clamp ring 16 includes an opening that is preferably defined by three segments 52 of the inner wall thereof, each segment 52 having an arcuate wall facing the outer surface of the split rear section 50 of the front slide 12 and which slopes radially inwardly from one end of each arcuate segment 52 to an opposite end. When the clamp ring 16 is turned in one rotational direction on the front slide 12, it compresses the split rear section 50 of the proximal end portion of the front slide 12. This, in turn, compresses the O-rings 40 within the bore of the proximal end portion of the front slide 12 and decreases the inner diameters of the openings in the O-rings 40.

When the endoscopic surgical instrument 2 of the present invention is mounted on an endoscope or arthroscope such that the tubular distal end portion of the endoscope or arthroscope passes through the bore of the front slide 12 and the central openings formed in the O-rings 40 and at least partially through the bore of the inner tube 8 mounted on the front slide 12, by rotating the clamp ring 16 in one direction on the front slide 12, the O-rings 40 are compressed about the tubular distal end portion of the endoscope or arthroscope to secure the endoscopic surgical instrument 2 to the endoscope or arthroscope and to maintain the position of the tubular distal end portion of the endoscope or arthroscope within the inner tube 8 of the surgical instrument 2 of the present invention. Turning the clamp ring 16 in an opposite direction will cause the O-rings 40 to release their hold on the endoscope or arthroscope allowing it to be removed from the surgical instrument 2.

The endoscopic surgical instrument 2 of the present invention further includes the cannula 30 which is, basically, a tubular member 54 having an internal lumen that extends from an enlarged diameter (when compared to that of the tubular member), cup-shaped member 56 that attaches to the front end of the back cannula 32, as will be described in greater detail. The tubular member 54 of the cannula 30 includes a flattened top wall 58, and a slot 60 formed through the thickness of the flattened top wall 58 and extending axially thereon over most or at least a portion of the longitudinal length thereof. The slot 60 is provided to allow a retractable blade 4 to project therefrom, as will be explained in greater detail. Preferably, the cannula 30, and at least the tubular member 54 thereof, is transparent so that an endoscope or arthroscope received by the surgical instrument 2 of the present invention and which passes through at least a portion of the lumen of the cannula 30 can view through the clear, transparent cannula 30 any tissue and other anatomical features at a surgical site when a surgeon is performing a procedure on a patient.

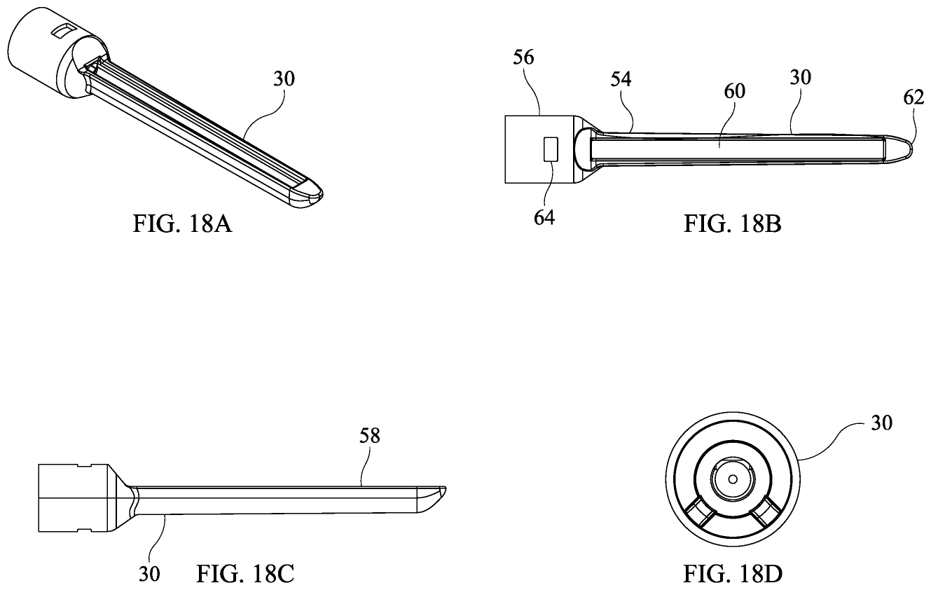

The cannula 30 is shown in various views in FIGS. 18A-18D of the drawings. The distal end of the cannula 30 is preferably closed and blunt, with a curved end, to define an obturator 62 thereat so that the cannula 30, when being positioned at a surgical site, will minimize any injury to the tissue that the cannula 30 comes in contact with. The retractable blade 4, as can be seen in FIGS. 2 and 3 of the drawings, is positioned at the distal end of the cannula 30, but slightly axially inwardly of the obturator end 62 thereof.

The cup-shaped proximal end 56 of the cannula 30 preferably includes two diametrically opposed slots or recesses 64 positioned on the outer side wall thereof. The slots 64 are provided to preferably fixedly mount the transparent cannula 30 to the back cannula 32 of the surgical instrument 2 of the present invention, as will now be described.

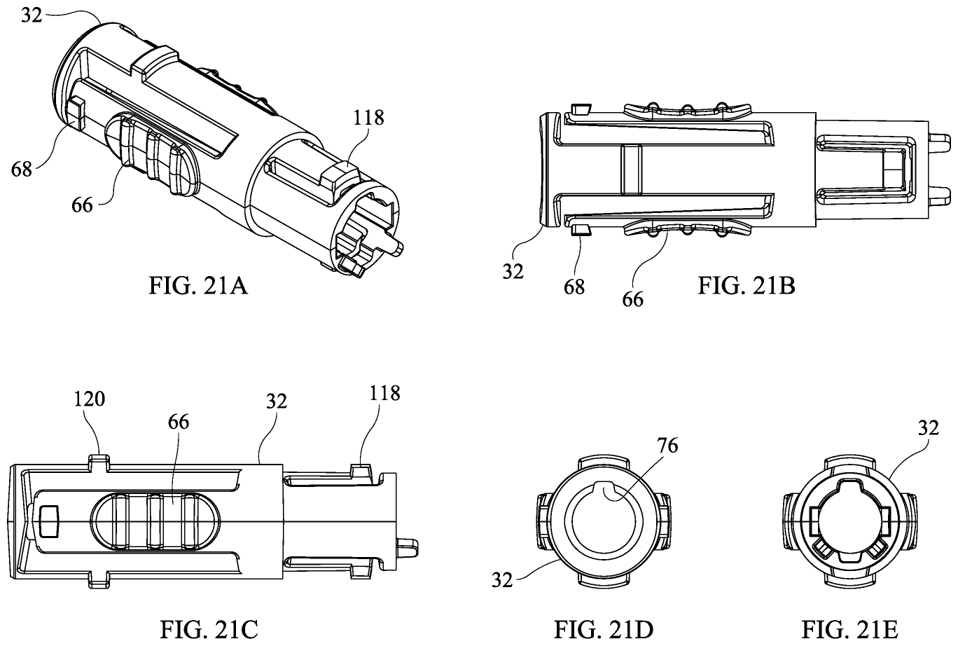

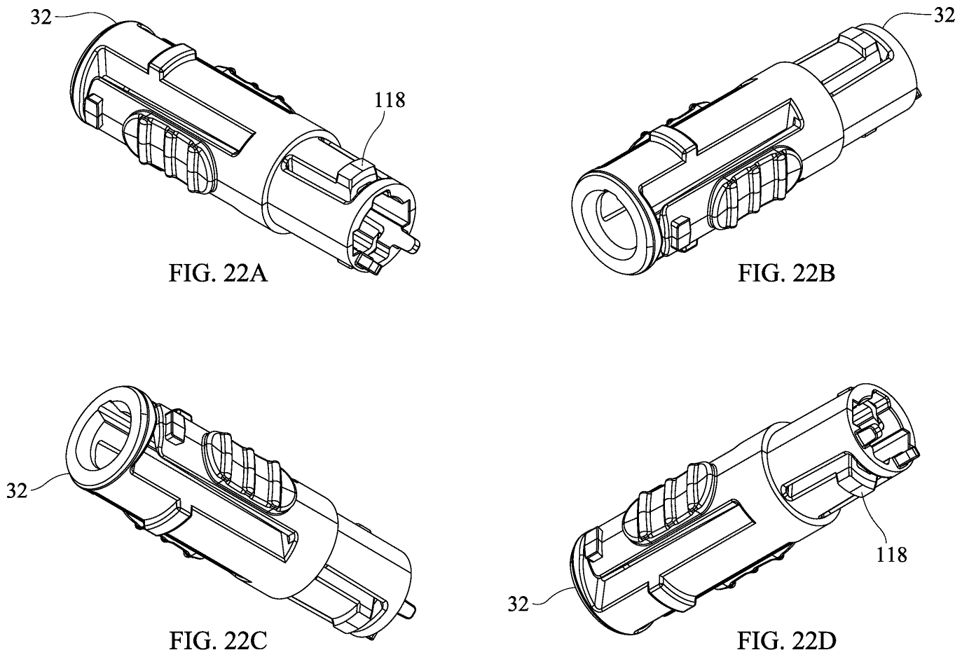

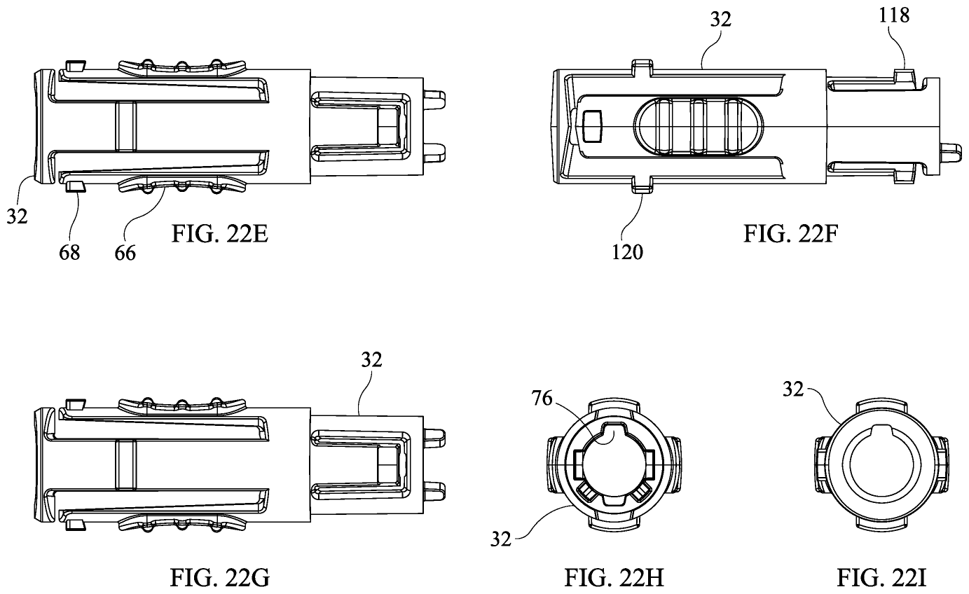

The back cannula 32 is generally cylindrical in shape and includes a central bore passing axially therethrough, as shown in the various views of FIGS. 21A-21E and 22A-22I of the drawings. The back cannula 32 is preferably removably mounted on the tubular distal end portion 46 of the front slide 12 as well as the main housing 18 of the surgical instrument 2.

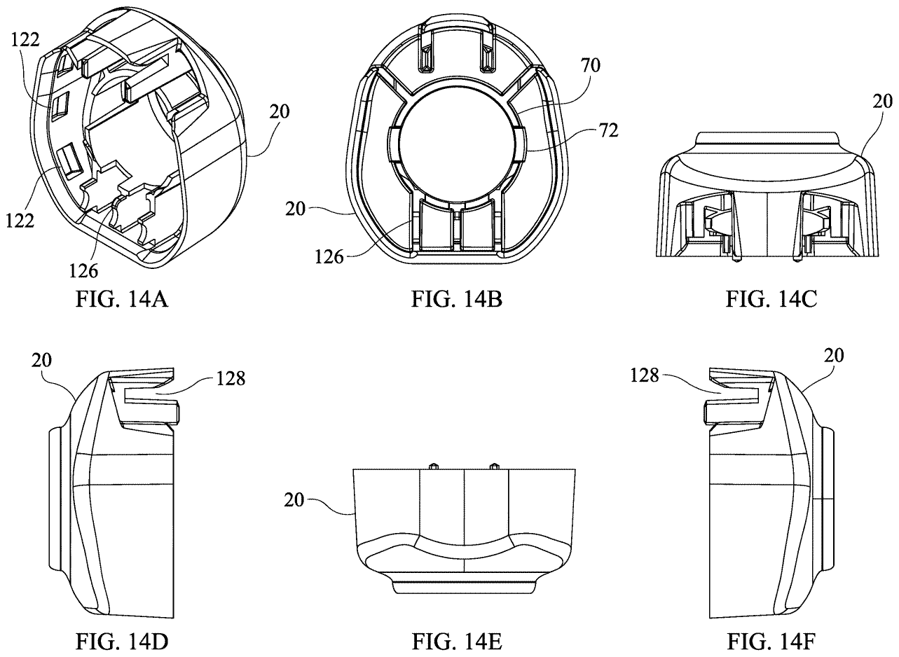

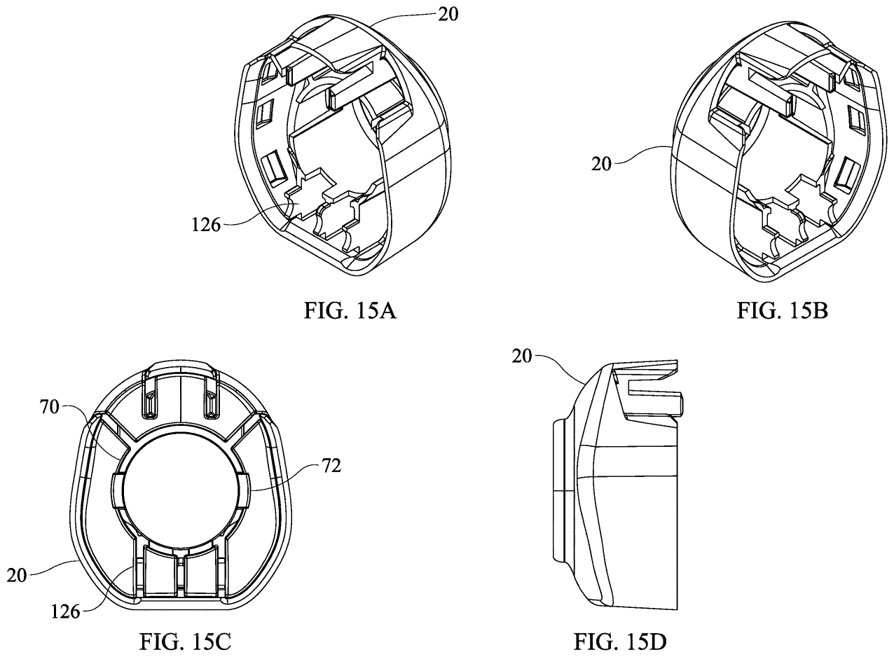

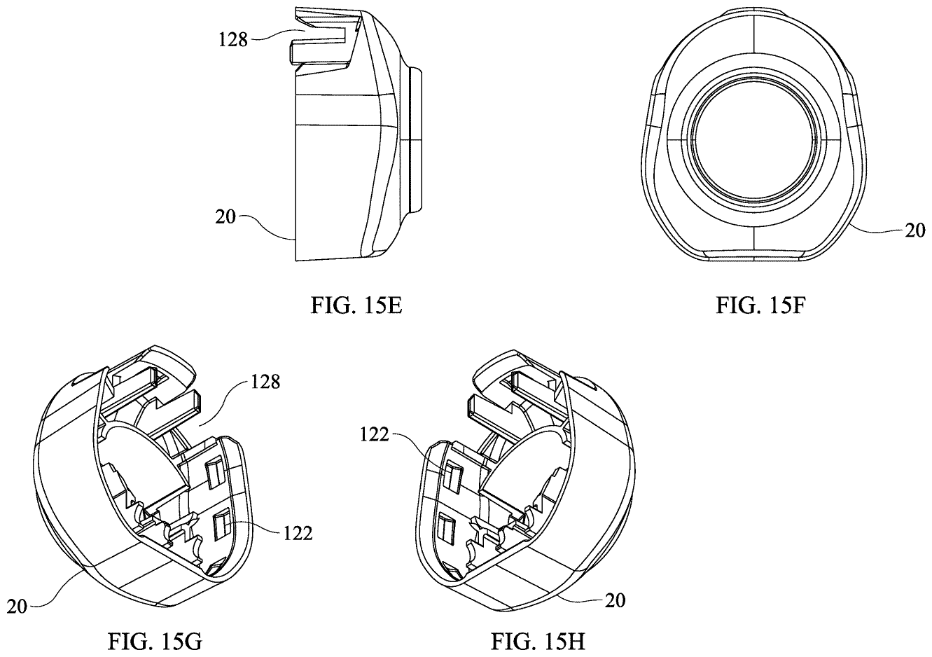

Again referring to FIGS. 21A-21E and 22A-22I of the drawings, it can be seen that the back cannula 32 has portions of the outer wall thereof formed as diametrically opposite resilient segments 66. Each segment is knurled with transverse spaced apart ridges extending outwardly from the outer surface thereof to facilitate locating and gripping the resilient segments 66 by a surgeon. At the distal free end of each resilient segment 66 is a barb 68. The proximal end portion of the back cannula 32 is received in the opening formed in the front housing section 20. As can be seen in the various views of the front housing section 20 shown in FIGS. 14A-14F and 15A-15H, the front housing section 20 includes an inner wall 70 surrounding and defining the opening therein, the inner wall 70 including two oppositely disposed slots 72. The barbs 68 of the two resilient segments 66 of the back cannula 32 are received in respective slots 72 of the front housing section 20 when the back cannula 32 is mated thereto. Thus, the front housing section 20 secures the back cannula 32 thereto but allows the back cannula 32 and transparent cannula 30 mounted thereon to be removed from the main housing 18 of the surgical instrument 2 by compressing the diametrically opposed resilient segments 66 of the back cannula 32. Such is an advantageous feature of the endoscopic surgical instrument 2 of the present invention. In some surgical procedures, it may be desirable to detach the cannula 30 and back cannula 32 from the surgical instrument 2 such that the slotted cannula 30 remains positioned at the surgical site while a surgeon performs a tissue cutting operation by having the retractable blade 4 projecting from the cannula slot 60 and by withdrawing the front slide 12 and inner tube 8 affixed thereto through the lumen of the cannula 30. The cutting blade 4 projecting through the slot 60 of the cannula 30 traverses a length of the slot 60 to sever tissue thereat until the blade 4 is retracted by the surgeon by releasing the lever 14.

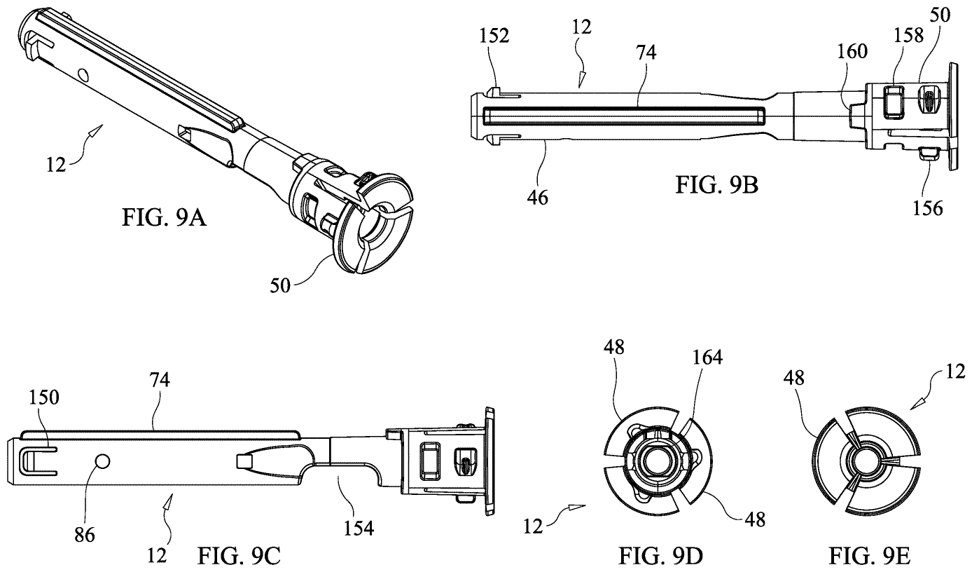

Reference should now be had to FIGS. 9A-9E which show various views of the front slide 12. A rib 74 extends at least partially along the axial length of the outer wall of the front slide 12 on the tubular distal end portion thereof. This axially extending rib 74 is received by an axially extending slot 76 formed in the inner wall of the back cannula 32 when the back cannula 32 is mounted on the tubular distal end portion of the front slide 12. The interaction between the rib 74 of the front slide 12 and the slot 76 of the back cannula 32 is to prevent the back cannula 32, and the transparent cannula 30 mounted thereon, from rotating on the front slide 12 so that the slot 60 of the cannula 30 through which the retractable cutting blade 4 selectively projects remains in alignment with the blade 4 and so that the cannula 30 stays in a desired position at a surgical site with respect to tissue or other anatomical features when a surgeon is performing a procedure on a patient.

The inner tube 8 is received by the lumen of the cannula 30 and is fixedly mounted on the front slide 12 and passes through the bore of the front slide 12. The inner tube 8 is shown in the various views of FIGS. 6A-6G of the drawings. Near the distal end of the inner tube 8 and within the bore thereof is disposed the retractable blade assembly 6. The cutting blade 4 of the retractable blade assembly 6 may extend out of and retract into the bore of the inner tube 8 through a slot 78 formed in the side wall of the inner tube 8. A cutout, defining a window 80, is also formed in the side wall of the inner tube 8. The camera or viewing sensor of an endoscope or arthroscope fitted onto the surgical instrument 2 of the present invention may be positioned to be in alignment with the window 80 formed in the inner tube 8 so that tissue or other anatomical features at a surgical site may be viewed using the endoscope or arthroscope during a surgical procedure.

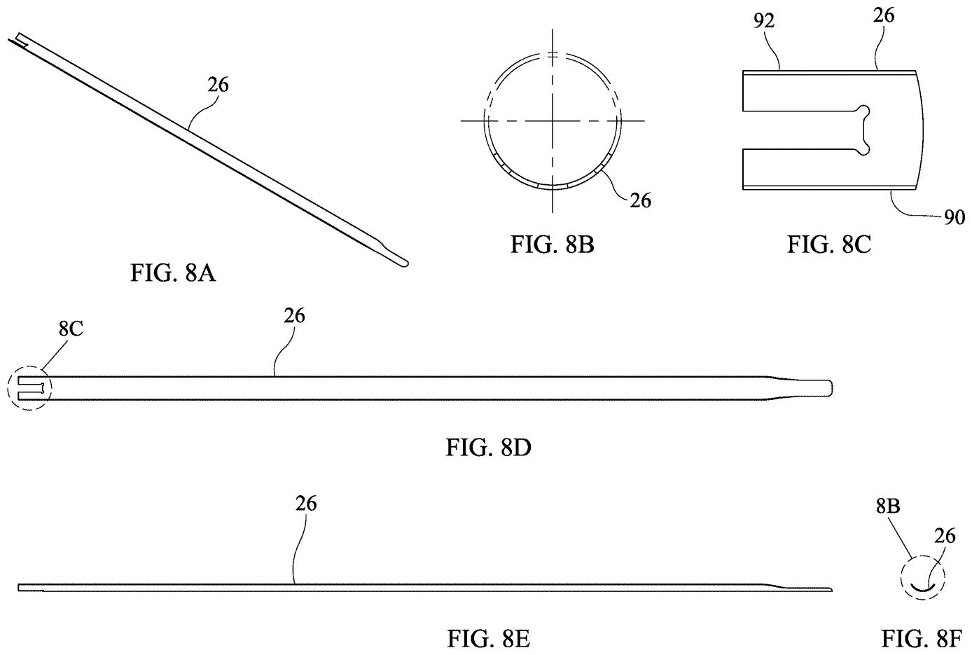

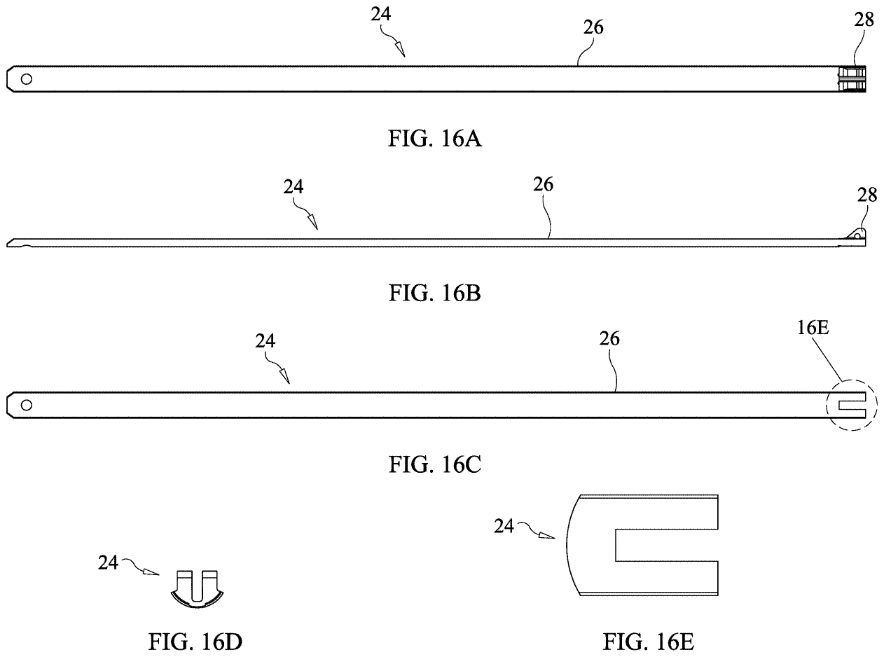

The projection of the cutting blade 4 through the slot 78 formed in the wall of the inner tube 8 and the slot 60 of the cannula 30, and the retraction of the blade 4 into the bore of the inner tube 8 is effected by the coupling of the retractable blade assembly 6 and the lever 14 through the inner slide member 26. The inner slide member 26 is shown in detail in the various views of FIGS. 8A-8F and FIGS. 16A-16E. The inner slide member 26 is particularly shaped so that it may reciprocatingly slide within the bore of the inner tube 8 when the lever 14 is moved in a forward position or a rearward position on the main housing 18 and without interfering with the tubular distal end portion of the endoscope or arthroscope which is also received by the bore of the inner tube 8.

Further details relating to the individual components of the endoscopic surgical instrument 2 of the present invention will now be described and reference should be had to the various views of the components shown in FIGS. 6A-6G, 7A-7F, 8A-8F, 9A-9E, 10A-10D, 11A-11D, 12A-12J, 13A-13C, 14A-14F, 15A-15H, 16A-16E, 17A-17D, 18A-18D, 19A-19F, 20A-20H, 21A-21E and 22A-22I.

FIGS. 6A-6G show one form of the inner tube 8 of the endoscopic surgical instrument 2 of the present invention in various views. As can be seen from these figures, the inner tube 8 is an elongated member having an internal bore extending axially therethrough. The inner tube 8 is received by the lumen of the transparent cannula 30. The proximal end portion of the inner tube 8 is mounted to the front slide 12 and is received by and affixed within the bore of the tubular distal end portion 46 of the front slide 12. The bore at the distal end portion of the inner tube 8 receives the pivot cap 10 forming part of the retractable blade assembly 6 to effectively close the distal end of the bore. A slot 78 is formed in the side wall of the inner tube 8 in alignment with the cutting blade 4 of the retractable blade assembly 6 and in alignment with the slot 60 of the transparent cannula 30 so that the cutting blade 4 may project outwardly from, and be retracted into, the bore of the inner tube 8 through the slot 78 of the inner tube 8.

Slightly axially inwardly from the distal end of the inner tube 8 is a window 80 defined by a cutaway portion of the side wall over about a 180 degree, or slightly greater, transverse portion of the side wall. Furthermore, a portion of the side wall of the inner tube 8 is cut at an angle to provide the window 80 with preferably about a 160 degree expanse in the axial direction of the inner tube 8. This window 80 is provided so that the distal end of an endoscope or arthroscope, which is angled at about 30 degrees, may view through this window 80 and through the clear, transparent cannula 30 any tissue or anatomical structure of a patient at a surgical site during a surgical procedure. More specifically, the tubular distal end portion of the endoscope or arthroscope is received by the bore of the inner tube 8 so that the viewing end thereof is positioned in proximity to the window 80 formed in the side wall of the inner tube 8.

A cutout 82 is also formed in the side wall of the inner tube 8 at the proximal end portion thereof and on the diametrically opposite side of the inner tube 8 from where the window 80 is located. This cutout 82 in the side wall of the inner tube 8 at the proximal axial end thereof is provided to receive the proximal end portion of the inner slide member 26 (shown in FIGS. 8A-8F) so that the proximal end portion of the inner slide member 26 may pass through the proximal cutout 82 and be affixed to the lever 14 to effect reciprocating movement of the inner slide member 26 within the inner tube 8 with pivotal movement of the lever 14 on the main housing 18 of the surgical instrument 2.

As also can be seen from FIGS. 6A and 6C, the inner tube 8 includes a bendable tab 84 cut into the side wall of the inner tube 8. When assembling the inner tube 8 and front slide 12 together, the inner tube 8 is received by the bore of the front slide 12 until the bendable tab 84 is in alignment with an opening 86 formed through the thickness of the tubular distal end portion of the front slide 12. The tab 84 of the inner tube 8 is bent outwardly from the outer surface thereof so that it is received by the opening 86 formed in the front slide 12 to secure the inner tube 8 to the front slide 12.

Furthermore, two parallel and spaced apart slots 88 are formed in an arcuate portion of the side wall of the inner tube 8 at the distal end portion thereof transversely on the inner tube 8. The slots 88 are provided so that the portion of the side wall of the inner tube 8 between the pair of slots 88 may be crimped inwardly to secure the pivot cap 10 forming part of the retractable blade assembly 6 onto the inner tube 8 at the distal end portion thereof.

The inner slide member 26 is shown in greater detail in the views of FIGS. 8A-8F of the drawings. The inner slide member 26 is an elongated member with a concave curvature in transverse cross-section. The transverse curvature of the inner slide member 26 is slightly less in its outer diameter than the inner diameter of the inner tube 8. For example, and as shown in FIG. 6G of the drawings, the inner diameter of the inner tube 8 is preferably about 0.178 inches. As shown in FIG. 8B of the drawings, the transverse curvature of the inner slide member 26 has an outer diameter of about 0.175 inches. Accordingly, the inner slide member 26 may be received within the bore of the inner tube 8 and be in close proximity to the inner surface of the side wall of the inner tube 8, leaving much of the bore of the inner tube 8 open to receive the tubular distal end portion of an endoscope or arthroscope to be received by the bore of the inner tube 8 without interfering with the placement of the endoscope or arthroscope within the inner tube 8 bore or constraining movement of the inner slide member 26 within the inner tube 8 while the endoscope or arthroscope's tubular distal end portion is situated in the axial bore of the inner tube 8.

The proximal end portion of the inner slide member 26 is narrowed and flattened widthwise with little or no curvature so that it may be bent about a cross pin 34 attached to the lever 14 to secure the proximal end of the inner slide member 26 to the lever 14. The opposite distal end of the inner slide member 26 is split in an axial direction to form a forked end 90. The forked distal end 90 of the inner slide member 26 engages the yoke 28 of the retractable blade assembly 6 and is attached thereto, with the slot formed between the prongs 92 of the forked end 90 of the inner slide member 26 receiving the transversely curved, axially extending mid-section of the underside of the yoke 28. As mentioned previously, since the proximal end of the inner slide member 26 is affixed to the lever 14 on the main housing 18 of the surgical instrument 2, and the opposite forked end 90 of the inner slide member 26 is operatively coupled to the retractable blade assembly 6, pivotal movement of the lever 14 on the main housing 18 of the surgical instrument 2 will effect reciprocating movement of the inner slide member 26 within the inner tube 8 and cause the cutting blade 4 to either project from or retract into the bore of the inner tube 8.

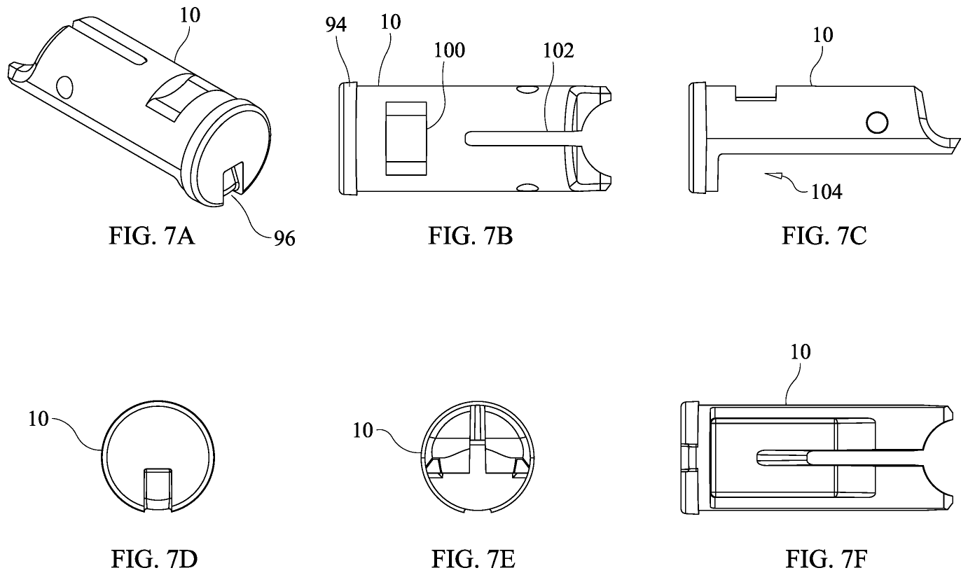

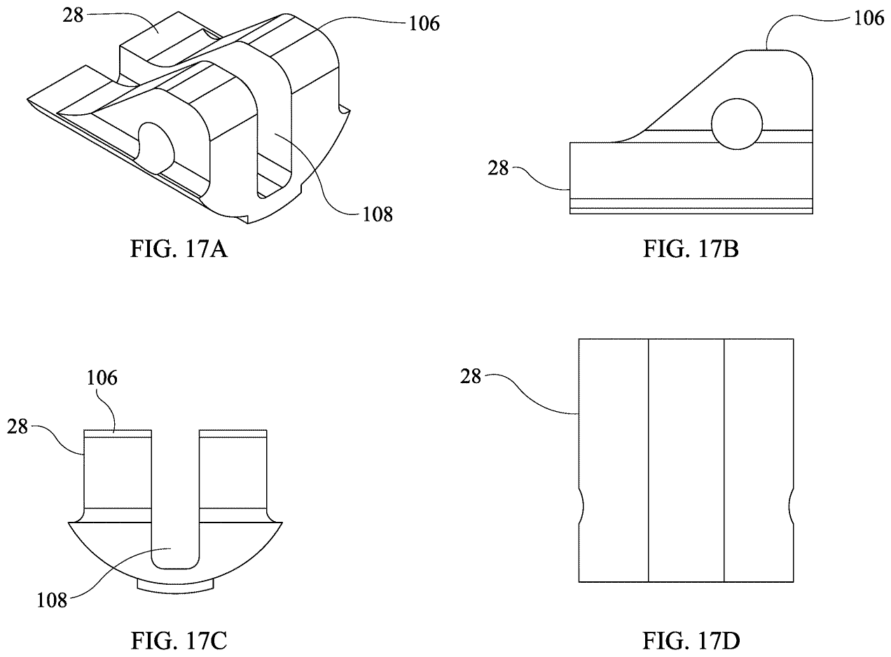

The retractable blade assembly 6 of the endoscopic surgical instrument 2 of the present invention will now be described in greater detail, and reference should be had to FIGS. 3A, 7A-7F, 16A-16E and 17A-17D of the drawings. More specifically, FIG. 3A shows the retractable blade assembly 6 within the inner tube 8 and lumen of the transparent cannula 30 and with the blade 4 projecting therefrom; FIGS. 7A-7F show in various views the pivot cap 10 of the retractable blade assembly 6; FIGS. 16A-16E show in various views the inner slide assembly 24, which includes the inner slide member 26 and the yoke 28 attached thereto; and FIGS. 17A-17D show in various views the yoke 28 of the retractable blade assembly 6.

Turning first to FIGS. 7A-7F, it will be seen that the pivot cap 10 of the surgical instrument 2 of the present invention is a generally cylindrical member that is situated at the distal end of the inner tube 8 and encloses the bore at the distal end of the tube. Thus, the pivot cap 10 is fixedly mounted on the distal axial end of the inner tube 8. The pivot cap 10 includes a disc-shaped distal end 94 which has a larger diameter relative to that of the proximal end portion of the pivot cap 10, the diameter of the disc-shaped distal end portion 94 preferably being substantially the same as the outer diameter of the side wall of the inner tube 8. The disc-shaped distal end portion 94 of the pivot cap 10 includes a notch 96 formed in the outer surface thereof. As can be seen from FIGS. 6A-6G of the drawings, the inner tube 8 at its distal end includes a tab 98 extending axially outwardly from the side wall of the inner tube 8. The pivot cap 10, when mounted on the distal end of the inner tube 8, is positioned such that the notch 96 on the pivot cap 10 is in alignment with the tab 98 on the inner tube 8. The tab 98 is bent upwardly and is received in the notch 96 of the pivot cap 10 to retain the pivot cap 10 on the inner tube 8. Furthermore, it can be seen from FIGS. 7A-7F that the pivot cap 10 also includes a transverse recess 100 formed in the outer wall thereof. This transverse recess 100 is provided so that a portion of the side wall of the inner tube 8 between the two transverse slots 88 described previously (see FIGS. 6B-6E of the drawings) may be crimped downwardly and received by the transverse recess 100 formed in the pivot cap 10 to further retain the pivot cap 10 to the inner tube 8 and to position the pivot cap 10 thereon in a particular relative position.

The proximal end portion of the pivot cap 10 up to the enlarged, disc-shaped distal end 94 thereof is received within the axial bore of the inner tube 8 at the distal end thereof. There is a slot 102 formed in the proximal end of the pivot cap 10 in which is received the retractable cutting blade 4 and from which the cutting blade 4 may extend and retract into. This slot 102 in the pivot cap 10 is positioned to be in alignment with the slot 78 formed through the thickness of the side wall of the inner tube 8 so that the blade 4 may project from and retract into both slots 78, 102. As will be described in greater detail, an actuator pin 42 passes through the cutting blade 4 and into the pivot cap 10 to direct movement of the cutting blade 4 into and out of the slot 102 of the pivot cap 10.

A lower portion of the generally cylindrical proximal end portion of the pivot pin 44 is removed to define an axial space 104. This space 104 is provided for receiving the yoke 28 forming part of the retractable blade assembly 6. The yoke 28 is mounted on the distal end of the inner slide member 26. The yoke 28 will now be further described, and reference should be had to FIGS. 17A-17D of the drawings which show the yoke 28 in various views.

The yoke 28 is structured similarly to a clevis, with two upstanding shoulders 106 which are parallel to each other and separated by a slot 108 in which at least a portion of the retractable cutting blade 4 is received. More specifically, the yoke 28 has the slot 108 formed in an upper surface thereof, which slot 108 is positioned to be in alignment with the slot 102 of the pivot cap 10.

There is a pivot pin 44 which passes transversely through the bottom of the yoke 28 through the two upstanding shoulders 106. This pivot pin 44 holds one end of the cutting blade 4 in place in the slot 108 of the yoke 28 and allows the cutting blade 4 to pivot thereon and within the slot 108 of the yoke 28. As mentioned previously, the actuator pin 42 passes transversely through the pivot cap 10 and through the slot 102 formed therein as well as through the thickness of the cutting blade 4.

The yoke 28 is joined to the forked distal end 90 of the inner slide member 26 by welding the two components 26, 28 together, thus forming the inner slide assembly 24 shown in the various views of FIGS. 16A-16E. Thus, the yoke 28 and inner slide member 26 reciprocatingly move axially within the bore of the inner tube 8 in response to pivotal movement of the lever 14 on the main housing 18 of the surgical instrument 2. The yoke 28 rides within the bore of the inner tube 8 at least partially underneath the pivot cap 10 and within the axial space 104 defined by the removal of the lower portion thereof from the proximal end portion of the pivot cap 10.

Reference should now be had to FIG. 3A of the drawings, which is an enlarged longitudinal cross-sectional view of the surgical instrument 2 of the present invention, and illustrating the retractable blade assembly 6 and the cutting blade 4 thereof. The retractable cutting blade 4 includes a forward facing sharpened edge 110 and a rearward facing sharpened edge 112. The blade 4 also includes an opening 114 formed transversely therethrough located generally opposite the cutting edges 110, 112 and at a lower portion of the blade 4. This opening 114 receives the pivot pin 44 of the yoke 28, and the pivot pin 44 secures the blade 4 to the yoke 28 but allows the blade 4 to pivot thereon.

The retractable cutting blade 4 further includes an arcuate slot 116 formed through the thickness thereof and at an upper portion thereof, nearer the sharpened edges 110, 112 than where the pivot pin 44 opening is located. This arcuate slot 116 is provided for receiving the actuator pin 42 that passes through the pivot cap 10. The actuator pin 42, when received by this arcuate slot 116 formed in the cutting blade 4, forces the cutting blade 4 to move within the slot 102 of the pivot cap 10 between an extended position (when the actuator pin 42 is at or near the bottom end of the arcuate slot 116) and a retracted position (when the actuator pin 42 is located at or near the upper end of the arcuate slot 116).

When the lever 14 on the main housing 18 is pivoted rearwardly against the bias of the compression spring 38 by the surgeon using finger pressure, the inner slide member 26, joined to the lever 14 and the yoke 28, causes the yoke 28 to move rearwardly axially within the inner tube 8. As a result, the cutting blade 4 pivots on the pivot pin 44 of the yoke 28 and is guided in its movement by the actuator pin 42 situated within the arcuate slot 116 formed therein. Since the pivot cap 10 is fixed within the inner tube 8, movement of yoke 28 in a direction axially towards the main housing 18 of the surgical instrument 2 causes the cutting blade 4 to move from a retracted state, with the actuator pin 42 being situated at or near the upper end of the arcuate slot 116 formed in the blade 4, to an extended state, with the actuator pin 42 being located at or near the lower end of the arcuate slot 116 formed in the blade 4, as shown in FIG. 3A, such that the blade 4 extends outwardly of the slot 102 formed in the pivot cap 10 as well as the aligned slot 78 formed in the inner tube 8.

When the lever 14 is released or moved to a forward position on the main housing 18 of the surgical instrument 2, biased in this direction by the compression spring 38, the compression spring 38 relaxes and causes the yoke 28 on the distal end of the inner slide member 26 to move in an axial direction towards the distal ends of the pivot cap 10 and inner tube 8, resulting in the cutting blade 4 being retracted within the slot 102 formed in the pivot cap 10 and the slot 78 formed in the inner tube 8, due to the blade 4 pivoting on the pivot pin 44 and being affixed to the slidable yoke 28, and with the actuator pin 42 now occupying the top end of the arcuate slot formed in the cutting blade 4. Accordingly, the retraction and extension of the cutting blade 4 through the slot 78 in the inner tube 8 and the slot 76 of the transparent cannula 30 may be easily controlled by the surgeon at any time during a surgical procedure. Thus, by controlling the position of the lever 14 on the main housing 18 of the surgical instrument 2, the surgeon may cause the cutting blade 4 to extend upwardly through the slot 78 of the inner tube 8 and the slot 76 of the transparent cannula 30 to effect the cutting of tissue during a surgical procedure.

The transparent cannula 30 of the surgical instrument 2 has been described previously in detail and is shown in the various views of FIGS. 18A-18D of the drawings. The transparent cannula 30 includes a tubular member 54 having an internal lumen that extends from an enlarged diameter (when compared to that of the tubular member), cup-shaped member 56 that attaches to the distal end of the back cannula 32. The tubular member 54 of the cannula 30 includes a flattened top wall 58, and a slot 76 formed through the thickness of the flattened top wall 58 and extending axially thereon over most or at least a portion of the longitudinal length thereof. The slot 76 is provided to allow the retractable cutting blade 4 to project therefrom, as explained previously. Preferably, the cannula 30, and at least the tubular member 54 thereof, is transparent so that an endoscope or arthroscope received by the surgical instrument 2 of the present invention and which passes through at least a portion of the lumen of the cannula 30 (through the inner tube 8) can view through the clear, transparent cannula 30 and the window 80 formed in the side wall of the inner tube 8 the tissue and other anatomical features at a surgical site when a surgeon is performing a procedure on a patient.

As further seen in FIGS. 18A-18D of the drawings, and as mentioned previously, the distal end of the transparent cannula 30 is preferably closed and blunt, with a curved end, to define an obturator 62 thereat so that the cannula 30, when being positioned at a surgical site, will minimize any injury to the tissue that the cannula 30 comes in contact with. The retractable blade 4, as can be seen in FIGS. 1-5, is positioned at the distal end of the transparent cannula 30, but slightly axially inwardly of the obturator end 62 thereof.

The cup-shaped proximal end 56 of the transparent cannula 30 preferably includes two diametrically opposed slots or recesses 64 positioned on a cylindrical side wall thereof. The slots 64 receive complementary-shaped projections 118 formed on the distal end portion of the back cannula 32 for attaching and securing the transparent cannula 30 to the back cannula 32.

The back cannula 32 is shown in the various views of FIGS. 21A-21E and FIGS. 22A-22I of the drawings. The back cannula 32 has an overall cylindrical shape, with the diameter of the distal end portion of the back cannula 32 being less than the diameter of the proximal end portion. As mentioned previously, the distal end portion of the back cannula 32 receives and secures thereto the transparent cannula 30, the cup-shaped proximal end 56 of the transparent cannula 30 being secured to the distal end portion of the back cannula 32 by the projections 118 of the back cannula 32 being received by the recesses 64 formed in the transparent cannula 30.

As also mentioned previously, the back cannula 32, with the transparent cannula 30 attached thereto, may be detached from the main housing 18 of the surgical instrument 2. As further mentioned previously, the proximal end portion of the back cannula 32 includes two diametrically opposed resilient segments 66. Each resilient segment 66 includes a barb 68 residing at the free end thereof. Furthermore, each resilient segment 66 may be knurled on the outer surface thereof such as with spaced apart, transversely disposed ribs or ridges to facilitate grasping of the resilient segments 66 and providing a tactile feel to the resilient segments 66 to facilitate location of the segments 66 on the back cannula 32 by a surgeon performing a surgical procedure. The resilient segments 66 may be squeezed together radially inwardly of the proximal end portion of the back cannula 32 to release the outwardly extending barbs 68 at the free ends of the resilient segments 66 from engagement with the main housing 18 of the surgical instrument 2. The resilient segments 66 of the proximal end portion of the back cannula 32 are biased to expand radially outwardly to secure the back cannula 32 and the transparent cannula 30 thereon in place on the main housing 18 of the surgical instrument 2. Thus, if required by a surgical procedure, the transparent cannula 30, and the back cannula 32 affixed thereto, may be disconnected from the main housing 18 of the surgical instrument 2 so that the transparent cannula 30 and back cannula 32 may remain in position at the surgical site, while the surgeon severs tissue at the site by withdrawing the cutting blade 4 or advancing the cutting blade 4 in either axial direction on the transparent cannula 30 which remains detached from the surgical instrument 2 and positioned at the surgical site.

The back cannula 32 includes at its proximal end portion two arcuate protrusions 120 extending outwardly from the outer surfaces of the back cannula 32 on diametrically opposite sides thereof. These protrusions 120 act as stops that abut against the front wall of the front housing section 20 to limit the distance the back cannula 32 may be inserted into the opening of the front housing section 20 and to help properly seat the barbs 68 of the resilient segments 66 into their respective slots 72 on the front housing section 20.

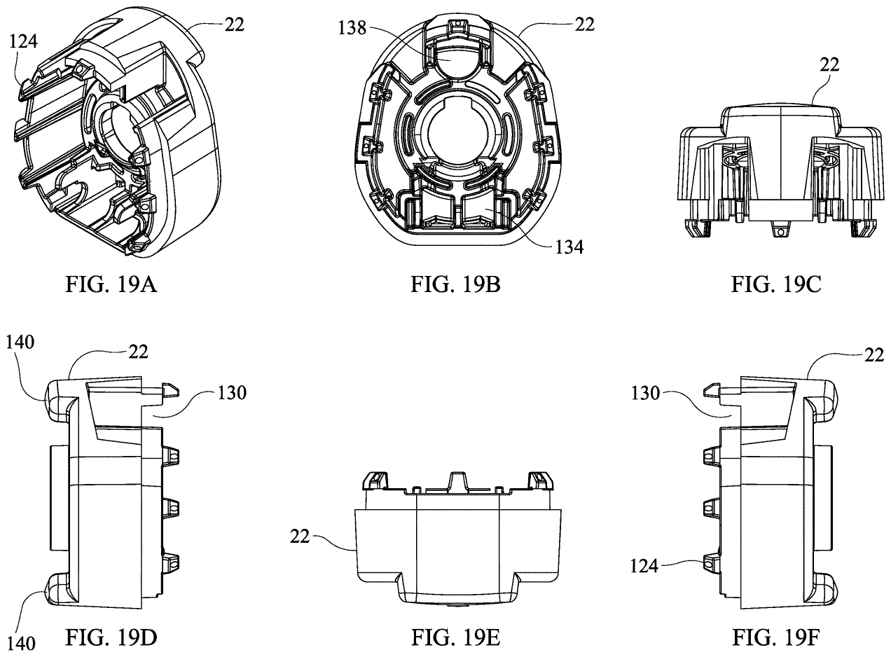

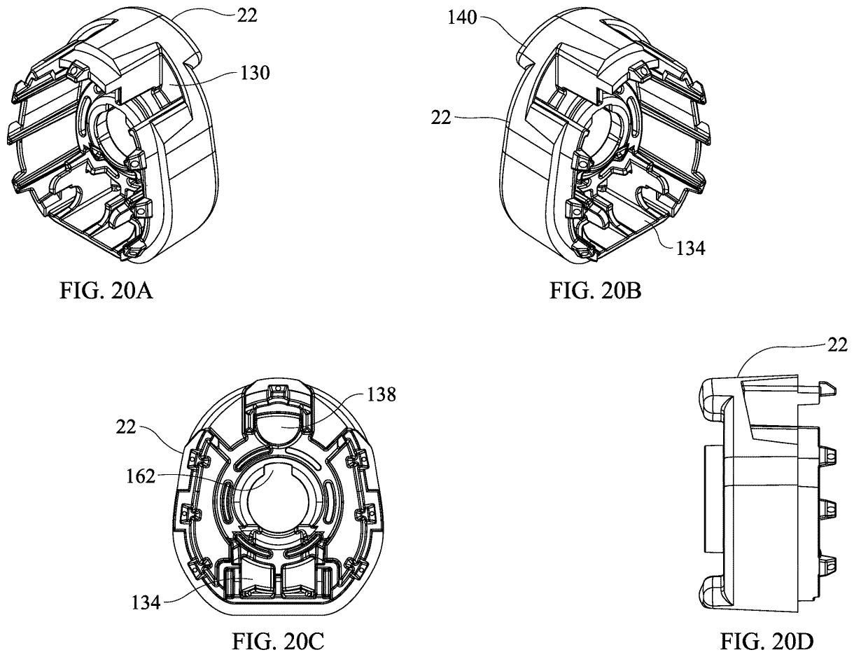

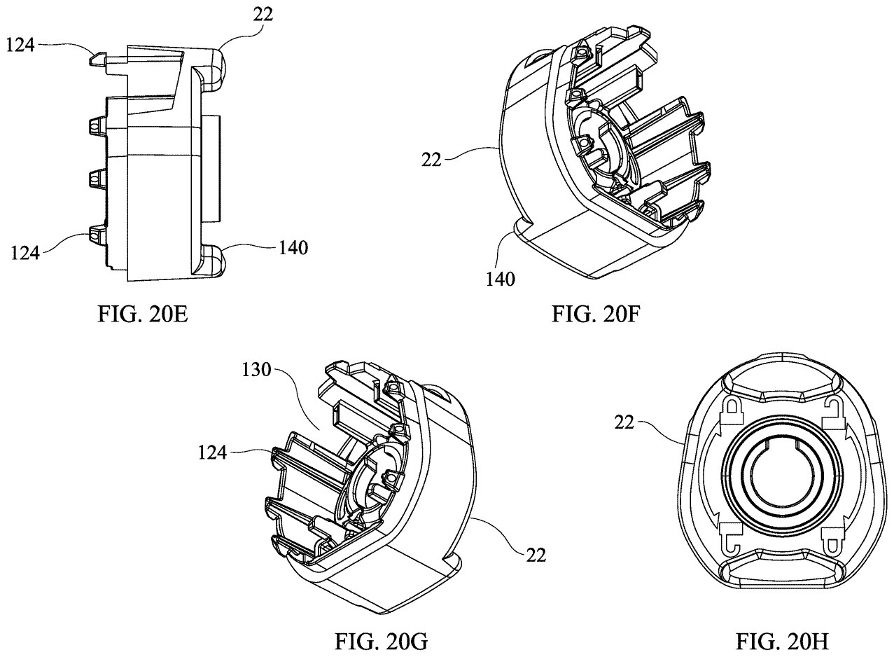

As also mentioned previously, the main housing 18 of the surgical instrument 2 of the present invention includes a front housing section 20 and a back housing section 22 which are mateable together to define the main housing 18. The front housing section 20 is shown in the various views of FIGS. 14A-14F and FIGS. 15A-15H. The back housing section 22 is shown in the various views of FIGS. 19A-19F and 20A-20H.

The front housing section 20 has a plurality of spaced apart recesses (or tabs) 122 formed about the periphery on the inside surface of the side wall of the front housing section 20. Such recesses (or tabs) 122 mate with corresponding tabs (or recesses) 124 formed on the side wall of the back housing section 22 about the periphery thereof to join the front housing section 20 to the back housing section 22. The front housing section 20 has an opening formed centrally therein through which is received the tubular distal end portion of the front slide 12 and the proximal end portion of the back cannula 32. Surrounding this opening is an inside wall 70 with two diametrically opposed slots 72 formed therein. The slots 72 receive the barbs 68 mounted on the resilient segments 66 of the back cannula 32 to detachably secure the back cannula 32 to the front housing section 20. The front housing section 20 also includes internally several spaced apart, axially projecting walls 126, the free ends of which are cup-shaped. As will be explained in greater detail, these walls 126 help secure the lower portion of the lever 14 within the interior of the back housing section 22 and to allow the lever 14 to pivot with respect thereto. Furthermore, the front housing section 20 includes separated slots 128 formed through the thickness of the top wall of the front housing section 20. The slots 128 are in alignment with corresponding slots 130 formed in the top wall of the back housing section 22 and receive arm portions 132 of the lever 14 that pass therethrough.

The back housing section 22, shown in FIGS. 19A-19F and 20A-20H of the drawings, includes a central opening formed through the rear wall thereof which receives the front slide 12 of the surgical instrument 2. The back housing section 22 and the front housing section 20, when mated together, define a cavity for receiving not only the compression spring 38 but also a portion of the lever 14. Near the bottom of the back housing section 22, within the interior cavity of the main housing 18, there are provided inner walls which define a receptacle 134 for receiving a lower portion of the lever 14 which defines an integrally formed pivot pin 136. As mentioned previously, the spaced apart, axially extending walls 126 of the front housing section 20, with their cup-shaped surfaces at the free ends thereof, engage this pivot pin 136 to maintain the pivot pin 136 within the receptacle 134 defined by the interior walls of the back housing section 22 when the front housing section 20 and back housing section 22 are mated together.

Also, within the interior of the back housing section 22 and at the upper portion thereof, axially extending walls at least partially define a receptacle 138 for receiving one axial end of the compression spring 38 which biases the lever 14 on the main housing 18 to be in a position in which the cutting blade 4 is in a retracted state within the bore of the inner tube 8. Furthermore, on the rear outer wall of the back housing section 22 extends diametrically opposite protrusions 140 which act as stops for the clamp ring 16, as will be described in greater detail.

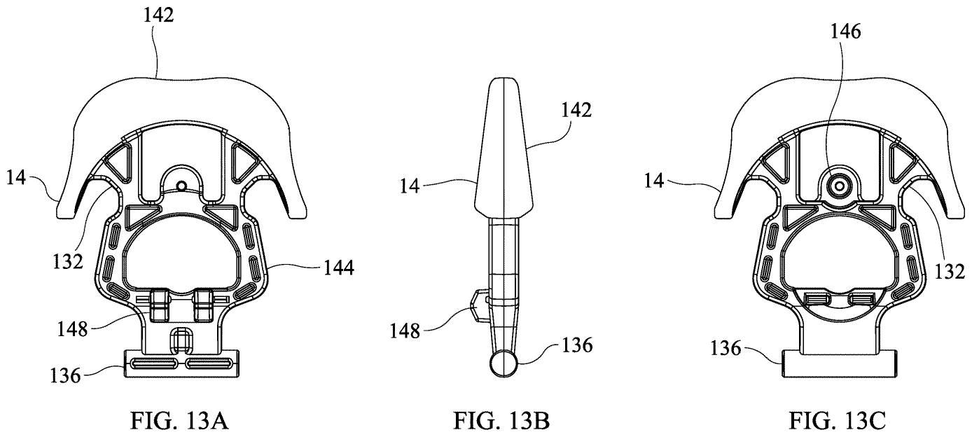

Now turning to FIGS. 13A-13C of the drawings, the lever 14 of the endoscopic surgical instrument 2 of the present invention is shown in the various views thereof. The lever 14 includes a main body having an upper portion 142 that resides over the main housing 18 of the surgical instrument 2 when the lever 14 is mounted thereon, a middle portion 144 having a central opening which is in alignment with the central opening of the back housing section 22 and the central opening of the front housing section 20, and a lower portion which is formed as a pivot pin 136, as described previously. Two spaced apart arms 132 connect the upper portion 142 with the middle portion 144 and pass through the slots 128, 130 formed in the top walls of the back housing section 22 and the front housing section 20. The upper portion 142 of the lever 14 is of sufficient width to reside over the full width of the top walls of the housing sections and to allow the surgeon using the endoscopic surgical instrument 2 of the present invention to exert finger pressure thereon to pivot the lever 14 on the main housing 18 to effect the retraction and extension of the cutting blade 4 through the slot 78 formed in the inner tube 8 and the slot 76 of the transparent cannula 30.

The rear surface of the lever 14 includes a post 146 for seating the other axial end of the compression spring 38. The front surface of the lever 14 includes two spaced apart hooks 148 which receive a cross pin 34. As mentioned previously, the proximal end portion of the inner slide member 26 extends through the cutout 82 formed in the proximal end portion of the inner tube 8 and is at least partially wrapped about the cross pin 34 to affix the proximal end portion of the inner slide member 26 to the lever 14 so that movement of the lever 14 on the main housing 18 of the surgical instrument 2 will effect axial movement of the inner slide member 26 within the bore of the inner tube 8, causing the cutting blade 4 to retract into or project from the inner tube 8 and the slot 76 of the cannula 30.

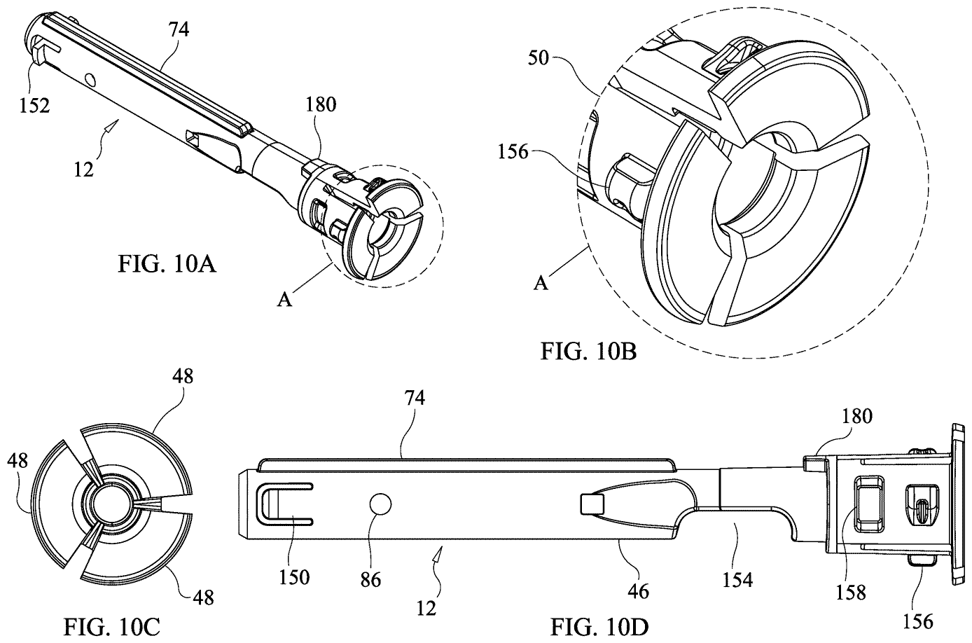

The front slide 12 of the endoscopic surgical instrument 2 of the present invention is shown in various views in FIGS. 9A-9E and 10A-10D of the drawings. The front slide 12 includes a tubular distal end portion 46 and a tubular proximal end portion, the proximal end portion having a diameter which is greater than that of the tubular distal end portion 46. As mentioned previously, there is a rib 74 that extends axially outwardly from the outer surface of the tubular distal end portion 46 of the front slide 12 over at least a portion of the axial length thereof. This rib 74 is received in a corresponding axially extending slot 76 formed in the inner surface of the back cannula 32. The rib 74 prevents the back cannula 32 and transparent cannula 30 connected thereto from rotating on the front slide 12 but allows axial movement of the back cannula 32 and transparent cannula 30 thereon if the back cannula 32 is detached from the main housing 18 of the surgical instrument 2.

The back cannula 32 and transparent cannula 30 mounted thereon may be completely removed from the free end of the distal end portion 46 of the front slide 12. To permit this, two diametrically opposed resilient members 150 are formed on the tubular side wall of the front slide 12 near the distal axial end thereof. Each resilient member 150 includes an outwardly extending barb 152. The barbs 152 on the front slide 12 are in alignment with and situated underneath the resilient segments 66 of the back cannula 32. When the surgeon presses the two resilient segments 66 on the back cannula 32 together, he can detach the back cannula 32 and transparent cannula 30 mounted thereon from the main housing 18 of the surgical instrument 2 to allow the inner tube 8 to slide axially within the lumen of the transparent cannula 30, for example, to effect a tissue cutting operation. The back cannula 32 and transparent cannula 30 will still remain affixed to the front slide 12 but axially movable thereon.

However, if the surgeon wishes to completely detach the back cannula 32 and the transparent cannula 30 mounted thereon from the surgical instrument 2, for example, to leave the transparent cannula 30 and back cannula 32 in place at the surgical site while removing the remainder of the surgical instrument 2 therefrom, he may do so by exerting greater pressure on the resilient segments 66 of the back cannula 32. Because the resilient members 150 of the front slide 12 are situated in alignment with the resilient segments 66 of the back cannula 32 on diametrically opposite sides of the front slide 12 and underneath the resilient segments 66 of the back cannula 32, greater pressure may be exerted on the resilient segments 66 of the back cannula 32 to depress the resilient members 150 on the tubular distal end portion 46 of the front slide 12 so that the barbs 152 on the resilient members 150 may pass through the rear opening formed in the proximal end portion of the back cannula 32. To remount the back cannula 32 and transparent cannula 30 attached thereto to the front slide 12, the rib 74 extending axially on the front slide 12 is positioned in alignment with the axially extending slot 76 formed in the inner surface of the back cannula 32, and the front slide 12 is inserted into the bore of the back cannula 32 through the rear opening thereof with slight force such that the inner wall defining the rear opening of the back cannula 32 rides over the inclined outer surfaces of the barbs 152 on the resilient members 150 of the front slide 12.

A cutout 154 is formed in the side wall of the front slide 12 at the proximal end portion thereof, which cutout is in alignment with the proximal cutout 82 formed in the inner tube 8. This allows the proximal end of the inner slide member 26 to extend through both cutouts 82, 154 and to be at least partially wrapped around the retaining cross pin 34 to secure the proximal end of the inner slide member 26 to the lever 14.

As also mentioned previously, and shown in FIGS. 9A-9E and 10A-10D of the drawings, the proximal end portion of the front slide 12 is split into preferably three resilient sections 48 which may be radially compressed inwardly. A ramp-like camming projection 156 extends outwardly from each resilient section 48. Additionally, an arcuate slot 158 is formed in the outer surface of each resilient section 48 preferably axially spaced from and in alignment with the ramp-like camming projections 156. The intended purpose for including such arcuate slots 158 is for injection molding purposes to minimize the differences in thickness in the cross sections of the front slide 12 when the front slide 12 is being molded.

As shown in FIGS. 9A-9C, 10A and 10D, the front slide 12 includes an arcuate protrusion 160 that extends axially from the larger diameter proximal end portion of the front slide 12 in a direction toward the smaller diameter, tubular distal end portion 46 of the front slide 12 and in alignment with the rib 74 formed on the tubular distal end portion 46 thereof. This arcuate protrusion 160 is received in a corresponding slot 162 formed in a smaller diameter inner ring wall of the back housing section 22 (see FIGS. 20C and 20H) to effect proper alignment of the front slide 12 in the back housing section 22 and to help prevent relative rotational movement between the main housing 18 and the front slide 12. Additionally, disposed within the bore of the front slide 12 at the proximal end thereof is a smaller diameter inner ring wall 164. This inner ring wall 164 is provided as a seat for the two resilient O-rings 40 that are received within the bore of the front slide 12 at the proximal end portion thereof.

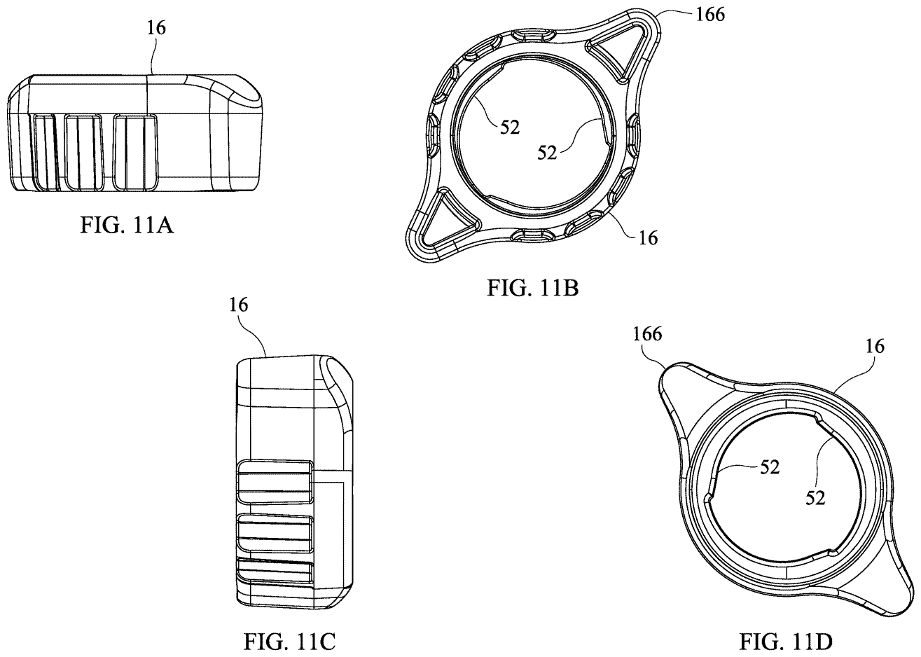

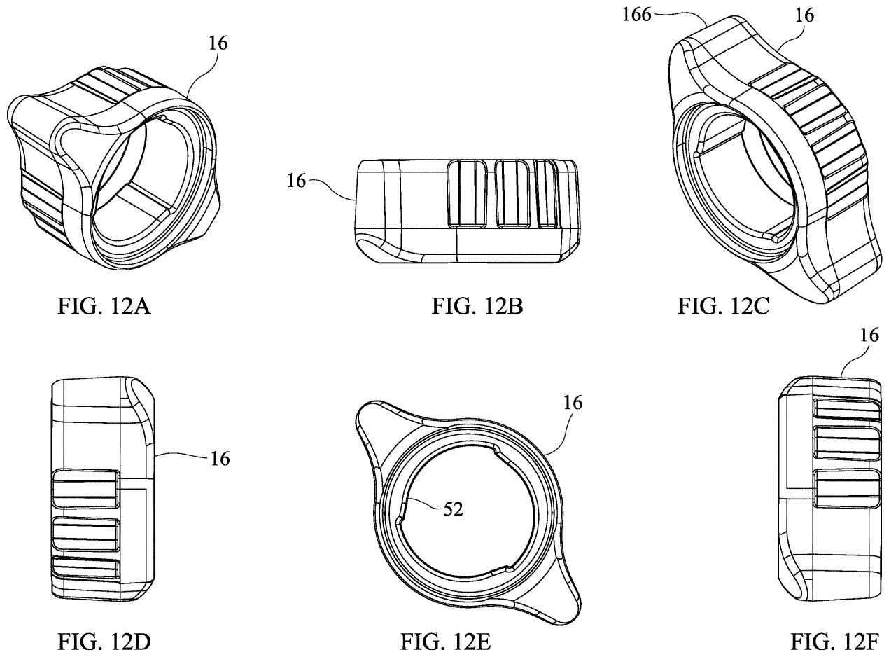

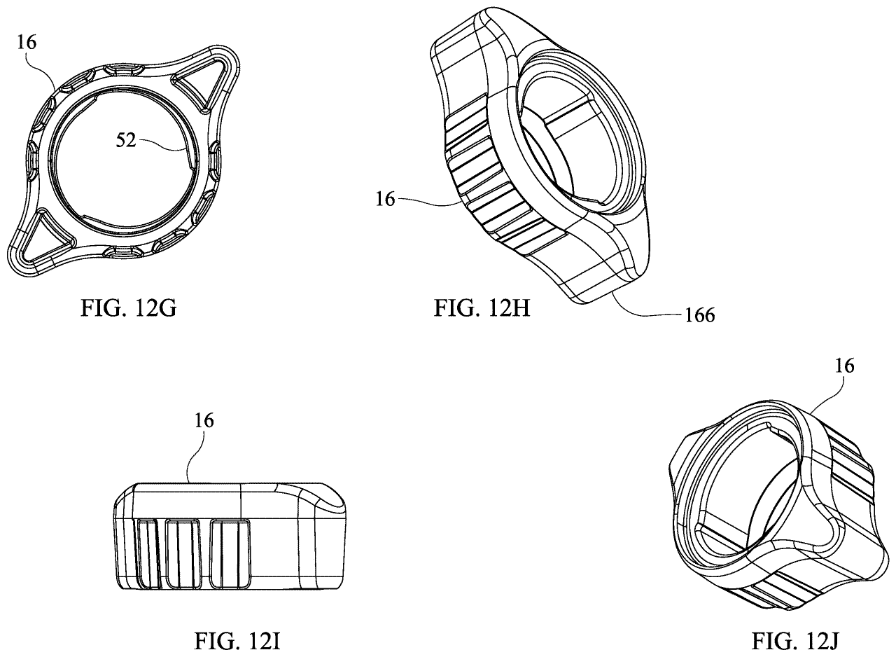

Turning now to FIGS. 11A-11D and FIGS. 12A-12J of the drawings, several views of the clamp ring 16 are shown. The clamp ring 16 is generally circular in overall shape, with two oppositely extending wings 166 to facilitate grasping and turning the clamp ring 16 by a surgeon. Also, the outer surface of the clamp ring 16 is preferably knurled to facilitate grasping the clamp ring 16. The two projections 140 on the rear wall of the back housing section 22 act as stops by engaging respective wings 166 on the clamp ring 16 to prevent the clamp ring 16 from being over-rotated in at least one direction on the front slide 12.

The clamp ring 16 is fitted over the proximal end portion of the front slide 12 and in alignment with the three compressible sections 48 thereof. The clamp ring 16 has an inner surface formed of three arcuate segments 52 which vary in diameter from the beginning to the arcuate end of each segment 52. For example, and as shown in FIG. 11B, which is a view of the rear face of the clamp ring 16, the internal wall of the clamp ring 16 over each arcuate segment decreases gradually in diameter in a clockwise direction. The three arcuate segments 52 of the inner wall of the clamp ring 16 engage corresponding ramp-like camming projections 156 of each of the three resilient split sections 48 of the proximal end portion of the front slide 12. Accordingly, when the clamp ring 16 is rotated in a clockwise direction, in one preferred embodiment of the present invention, the three arcuate segments 52 of the inner wall of the clamp ring 16 will engage and tighten down on the ramp-like camming projections 156, causing the split resilient sections 48 of the proximal end portion of the front slide 12 to compress radially inwardly of the bore of the front slide 12. As mentioned previously, the pair of O-rings 40 is situated within the bore at a proximal end portion of the front slide 12, and the O-rings 40 will also compress about the tubular distal end portion of an endoscope or arthroscope received through the bore of the front slide 12 and the openings in the O-rings 40 to hold the endoscope or arthroscope in a desired axial or rotational position within the surgical instrument 2 of the present invention, for example, with the viewing end thereof in alignment with the window 80 formed in the inner tube 8. Rotating the clamp ring 16 in the opposite direction on the front slide 12 will cause the resilient split sections 48 of the proximal end portion of the front slide 12 to expand, as well as the O-rings 40 situated within the bore of the front slide 12, so that the endoscope or arthroscope may be removed from the bore of the front slide 12 of the surgical instrument 2.

In preparing the surgical instrument 2 of the present invention for a surgical procedure, the distal end of an endoscope or arthroscope is inserted into the rear axial opening leading to the bore of the front slide 12 until the viewing end thereof is positioned in alignment with the window 80 formed through the side wall of the inner tube 8. The clamp ring 16 is then turned to compress the split proximal end of the front slide 12 to compress the three sections 48 thereof and the O-rings 40 therein through which the endoscope or arthroscope passes to tighten about the endoscope or arthroscope to secure the endoscope or arthroscope to the surgical instrument 2 of the present invention.

During transport of the surgical instrument 2, or prior to its use, or when the instrument 2 is discarded after a surgical procedure, a protective end cap 36 (see FIG. 5) is securely fitted onto the distal end portion of the transparent cannula 30. The axial length of the end cap 36 is preferably chosen to cover a portion of the cannula 30 where the retractable cutting blade assembly 6 within the inner tube 8 resides so as to prevent exposure of the cutting blade 4 through the cannula slot 60 should the lever 14 be inadvertently moved to a blade extended position.

Although illustrative embodiments of the present invention have been described herein with reference to the accompanying drawings, it is to be understood that the invention is not limited to those precise embodiments, and that various other changes and modifications may be effected therein by one skilled in the art without departing from the scope or spirit of the invention.

What is claimed is:

1. An endoscopic surgical instrument, which comprises:
a main housing;
a spring-biased lever mounted on the main housing and movable pivotally with respect thereto between a first position thereon and a second position thereon opposite the first position;
a spring operatively coupled to the lever and biasing the lever in the second position;
a generally tubular front slide section which passes through the main housing and having a bore in which is receivable a tubular distal end portion of an endoscope or arthroscope for viewing a surgical site by a surgeon performing a surgical procedure on a patient, the endoscope or arthroscope having a camera or viewing sensor disposed near the tubular distal end portion thereof;
a transparent, slotted cannula having a lumen;
a back cannula section to which the transparent cannula is affixed, the back cannula section being removably mounted to the main housing;
a clamp ring which is partially rotatable on the front slide section to secure the endoscope or arthroscope in place and in a desired position on and relative to the surgical instrument;
an inner tube having a distal end, the inner tube extending through the lumen of the transparent cannula and including a slot formed through a wall thereof near the distal end of the inner tube, the slot of the inner tube being in alignment with the slot of the transparent cannula, the inner tube having an axial bore, the tubular distal end portion of the endoscope or arthroscope being receivable by the axial bore of the inner tube and disposable in a desired placement and positioning therein, the inner tube having a cutout formed in the wall thereof near the distal end thereof, which cutout defines a window through which, in addition to the transparent cannula, the camera or viewing sensor of the endoscope or arthroscope may view the surgical site during the surgical procedure;
an elongated, inner slide member residing within the axial bore of the inner tube and axially movable therein, the inner slide member being operatively coupled to the lever and reciprocatingly movable within the bore of the inner tube in response to pivotal movement of the lever, the inner slide member being configured so as not to interfere with the placement and positioning of the tubular distal end portion of the endoscope or arthroscope when the distal end portion of the endoscope or arthroscope is situated within the bore of the inner tube; and
a blade assembly disposed within the bore of the inner tube near the distal end of the inner tube, the blade assembly being operatively coupled to the inner slide member and having a retractable cutting blade;
wherein, when the lever on the main housing is pivoted against the bias of the spring from the second position to the first position, the inner slide member also moves in a first axial direction within the inner tube, causing the cutting blade to project not only through the slot formed in the wall of the inner tube but also through the slot of the cannula to effect tissue cutting during the surgical procedure; and
wherein, when the lever is moved to the second position opposite the first position, the inner slide member concomitantly moves in a second axial direction opposite the first direction to cause the cutting blade to safely retract into the bore of the inner tube without projecting therefrom and from the slotted cannula.

2. An endoscopic surgical instrument as defined by claim 1, wherein the inner slide member has a curved shape in transverse cross-section so as so as not to interfere with the tubular distal end portion of the endoscope or arthroscope when the distal end portion of the endoscope or arthroscope is situated within the bore of the inner tube.

3. An endoscopic surgical instrument as defined by claim 1, wherein the main housing has a front housing section and a back housing section which is mateable with the front housing section.
